(19) United States
(12) Patent Application Publication
(10) Pub. No.: US 2013/0045236 A1
(43) Pub. Date: Feb. 21, 2013

(54) DIAGNOSTIC AND THERAPEUTIC USES OF SOLUBLE FC-EPSILON RECEPTOR I FOR IGE-MEDIATED DISORDERS

(71) Applicants: Elisabeth-Edda Fiebiger, Cambridge, MA (US); Wayne I. Lencer, Jamaica Plain, MA (US)

(72) Inventors: Elisabeth-Edda Fiebiger, Cambridge, MA (US); Wayne I. Lencer, Jamaica Plain, MA (US)

(21) Appl. No.: 13/496,786

(22) PCT Filed: Sep. 17, 2010
(86) PCT No.: PCT/US10/02538

§ 371 Date: Oct. 28, 2012

Related U.S. Application Data
(60) Provisional application No. 61/243,519, filed on Sep. 17, 2009.

(51) Int. Cl. G01N 33/566; A61K 38/17; A61P 37/02; A61P 11/06; A61P 35/02; A61P 1/00; A61P 17/00; A61P 33/00; A61P 29/00; C12M 1/34; A61P 37/08

(52) U.S. Cl. 424/278.1; 435/7.92; 436/501; 435/287.2; 422/69

(57) ABSTRACT

The present disclosure is based in part on the finding that sFcεRI is a novel biomarker for IgE-mediated disorders. Methods for diagnosing, treating and/or monitoring IgE-mediated disorders are also described. The disclosure further provides assays to detect sFcεRI in a sample.

### RELATED APPLICATIONS

This application claims the benefit of the filing date of U.S. provisional patent application 61/243,519, filed Sep. 17, 2009 entitled “DIAGNOSTIC AND THERAPEUTIC USES OF SOLUBLE Fc-EPSILON RECEPTOR I FOR IgE-MEDIATED DISORDERS”. The entire teachings and contents of the referenced provisional applications are incorporated herein by reference.

### FIELD OF THE DISCLOSURE

The present disclosure relates to diagnosing, monitoring and treating IgE-mediated disorders, such as allergic conditions.

### BACKGROUND OF THE DISCLOSURE

The overall incidence of allergies is increasing at a striking rate in developed countries. Moderate estimates suggest that one out of five individuals in the Western World suffers from some form of allergic condition. Allergic patients are commonly, but not always, characterized by high serum IgE and high IgE-receptor levels on effector cells of the innate and adaptive immune system. Humans express three different IgE-receptors: CD23, galectin 3, and Fc-epsilon receptor I, or FcεRI. CD23, also known as Fc-epsilon receptor II, or FcεRII, is a low affinity IgE receptor and a classical IgE-trafficking structure. Galectin 3 is another low affinity IgE receptor and its functions for the allergic response is poorly defined. IgE-antigen complexes induce activation of mast cells and basophils during the acute phase of the allergic response via FcεRI, the high-affinity receptor for IgE. In rodents, FcεRI is exclusively expressed as a tetrameric receptor composed of the ligand-binding α-chain, one β-chain and a pair of disulfide-linked γ-subunits on the surface of basophils and mast cells. By comparison, in the absence of β-transcripts, humans can express a trimeric version of FcεRI on eosinophils and antigen presenting cells, such as dendritic cells and Langerhans cells.

Currently, there are several major techniques used in the diagnosis of allergic disease, including skin tests, assays of IgE serum levels, and histamine release tests. Skin tests have represented the primary diagnostic tool in allergy since their introduction in 1865. The classical skin test in atopy is the Type I wheal and flare reaction in which antigen introduced into the skin leads to the release of preformed mediators, increased vascular permeability, local edema and itching. Such skin tests can provide useful confirmatory evidence for a diagnosis of specific allergy that has been made on clinical grounds. However, when improperly performed, skin tests can lead to falsely positive or negative results. The main limitation of the skin test is that a positive reaction does not necessarily mean that the disease is allergic in nature, as some non-allergic individuals have specific IgE antibodies that produce a wheal and flare reaction to the skin test without any allergic symptoms.

The IgE-mediated false positive phenomenon observed in skin tests is not observed in in vitro methods for assaying allergen-specific IgE in patient serum (see Homburger and Katzmann, “Methods in Laboratory Immunology: Principles and Interpretation of Laboratory Tests for Allergy,” in Allergy Principles and Practice, Middleton et al., eds, Mosby, pub., 4th Edition, vol. 1, chap. 21, pp. 554-572(1.993)). Typically, allergen-specific IgE levels are measured by a radioallergosorbent test (RAST) wherein a patient's serum is incubated with antigen-coated sorbent particles, followed by detection of the specific. IgE bound to antigen with labeled antibody (see, e.g., Schellenberg et al., J. Immunol., 115: 1577-1583 (1975)).

Total serum IgE levels are also used in the diagnosis of allergy. Total IgE levels can be measured by radioimmunoassay or immunometric assay methods as described by Homburger and Katzmann, supra. IgE levels are often raised in allergic disease and grossly elevated in parasitic infestations. When assessing children or adults for the presence of allergic (atopic) disease, a raised level of IgE aids the diagnosis, although a normal total IgE level does not exclude an allergic condition or tendency. The determination of total IgE alone will not predict an allergic state as there are genetic and environmental factors which play an important part in the production of clinical symptoms. The value of serum IgE level in allergy diagnosis is also limited by the wide range of IgE serum concentrations in healthy individuals. The frequency distribution of IgE concentrations in healthy adults is markedly skewed with wide 95 percentile limits and a disproportionate number of low IgE values. Accordingly, in calculating the 95 percentile limits of normal IgE levels most investigators treat their data by logarithmic transformation, which yields upper limits for normal serum IgE that are very high when compared with arithmetic means. These high upper limits for normal serum IgE diminish the diagnostic value of the serum IgE test in screening for clinical allergy. The use of serum IgE levels as an indication of an allergic condition is further complicated by the observation that some patients who present clinical symptoms for an allergic condition do not exhibit elevated serum IgE levels. In these “allergic” subjects, confirmation of the diagnosis presents a challenge.

Histamine release tests provide a means to detect functional, allergen-specific IgE in patient serum. Typically, histamine release tests imitate the allergen-specific reaction as it occurs in the patient (see, e.g., under van der Zee et al., J. Allergy Clin. Immunol., 82: 270-281 (1988)). This response has been generated in vitro by mixing a patient's blood with different allergens and later measuring the amount of histamine released during each of the subsequent allergic reactions. In vitro histamine release assays originally required the isolation of leukocytes from whole blood and/or various extractions of free histamine. Leukocyte histamine release tests were subsequently refined and automated to avoid cell isolation and histamine extraction (see, e.g., Siraganian et al., J. Allergy Clin. Immunol., 57: 525-540 (1976)). At present, commercially available leukocyte histamine release testing kits permit up to 100 separate determinations with 2.5 ml of whole blood. However, blood samples cannot be stored for more than 24 hours prior to assay. In addition, the tests produce false positive results due to non-specific histamine release produced by toxicity of the allergen extracts or other factors. Also, a quality control study has reported considerable inter-laboratory variability in the measurement of histamine (Gleich and Hull, J. Allergy Clin. Immunol., 66: 295-298 (1980)).

In a minority of subjects with allergic symptoms, positive skin tests and clearly detectable IgE antibodies, no in vitro histamine release can be obtained from the subjects' basophil leukocytes with allergen. This phenomenon makes it impossible to interpret the results of a histamine release test if positive controls are not available and limits the usefulness of the test in diagnosing allergic disease. Levy and Osler, J. Immunol., 99: 1062-1067 (1967) reported that leukocytes from only 20 to 30% of non-allergic individuals exhibit histamine release upon passive sensitization with allergen-specific IgE followed by allergen challenge in vitro. Ishizaka et al., J. Immunol., 111: 500-511 (1973) expanded the usefulness of the test by showing that the incubation of leukocytes with deuterium oxide (D₂O) enhanced the histamine release induced by passive sensitization of leukocytes with anti-ragweed serum and challenge with ragweed antigen. Prahl et al., Allergy, 43: 442-448 (1988) reported the passive sensitization of isolated, IgE-deprived leukocytes from non-allergic individuals with serum from a non-releasing allergic patient followed by allergen-induced histamine release. However, the Prahl et al. method requires isolation of control leukocytes from the whole blood of a non-allergic donor followed by removal of IgE bound to the donor cells. Additionally, the Levy et al., Ishizaka et al., and Prahl et al. procedures are subject to the same histamine assay variation that limits the usefulness of the other histamine-release tests described above.

Clearly, it is of great interest to establish reliable biomarkers for facilitating the diagnosis of clinical conditions such as allergy. Such biomarkers are important in the clinical development of effective therapeutics. A biomarker can be an indicator of normal biological processes, disease processes, or pharmacological responses to therapeutic intervention. Their role ranges from stratifying the patient population in helping to identify responders versus non-responders to certain therapeutics, to determining the efficacy of the therapeutic regimen. Biomarkers can be a valuable tool in making better decisions that will reduce the cost for drug development and enable therapeutics to reach the right patient population faster.

As discussed above, IgE-mediated disorders are very heterogeneous with respect to clinical symptoms and responsiveness to particular therapies. Thus, biomarkers that can aid in identifying a subpopulation of subjects having or is at risk of having a certain condition for predicting responsiveness to a certain therapeutic and/or predisposition for developing a certain condition would be useful for developing more effective treatment.

### SUMMARY OF THE DISCLOSURE

The present disclosure is at least in part based on the discovery that soluble alpha chains of Fc receptors can be used as in vivo marker of IgE-mediated activation of the immune system. Data presented herein support the notion that IgE-mediated cell activation induces the release of a soluble form of the FcεRI alpha chain (sFcεRI). This sFcεRI is found free and/or complexed with its ligand IgE in human serum. Due to the high affinity of ligand interaction characteristic for this IgE-receptor, it is believed that sFcεRI may be a potent regulator of IgE responses in vivo.

Based on these findings, in one aspect, the present disclosure provides a method for diagnosing an IgE-mediated disorder in a subject. Described herein are methods of using a convenient, reproducible and widely applicable test for the diagnosis of IgE-mediated disorders, by detecting or measuring sFcεRI. The method comprises: (i) detecting or measuring a level of sFcεRI in a sample from a subject; (ii) comparing the level of sFcεRI in the sample to a predetermined value; and, (iii) if the level of sFcεRI in the sample is above the predetermined value, identifying the subject as having or being at risk of having an IgE-mediated disorder. The sFcεRI may be free or complexed to IgE.

In some embodiments, the IgE-mediated disorder may include one or more of the following: esophagitis (e.g., eosinophilic esophagitis or EoE), gastroenteritis (e.g., eosinophilic gastroenteritis or EoG), hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis and necrotizing enterocolitis.

In some embodiments, the subject may be a human subject or a non-human subject. In some embodiments, the subject has a normal level of serum IgE.

The sample may be blood, serum, plasma, lymph, saliva or urine.

Another aspect of the disclosure involves a method of diagnosing an IgE-mediated disorder in a subject. The method comprises: (i) comparing a level of sFcεRI in a biological sample from the subject to a predetermined value; and, (ii) if the level of sFcεRI in the biological sample is above the predetermined value, identifying the subject as having or being at risk of having an IgE-mediated disorder. The sFcεRI may be free or complexed to IgE.

In certain embodiments, the IgE-mediated disorder may include but is not limited to: esophagitis (e.g., eosinophilic esophagitis or EoE), gastroenteritis (e.g., eosinophilic gastroenteritis or EoG), hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis or necrotizing enterocolitis.

In some embodiments, the subject may be a human subject or a non-human subject. In some embodiments, the subject has a normal level of serum IgE.

The sample may be blood, serum, plasma, lymph, saliva or urine.

A further aspect of the disclosure is directed to a method of evaluating the efficacy of a therapy for an IgE-mediated disorder in a subject. The method comprises: (i) detecting or measuring a level of sFcεRI in a sample from a subject having or at risk of having an IgE-mediated disorder before a therapy for the disorder; (ii) detecting or measuring a level of sFcεRI in a sample from a subject having or at risk of having an IgE-mediated disorder after the therapy for the disorder; and (iii) comparing the level of sFcεRI in the samples before and after the therapy, wherein a decrease in the level of sFcεRI in the sample after the therapy relative to the sample before the therapy indicates that the subject is responsive to the therapy. The sFcεRI may be free or complexed to IgE.

In some embodiments, an increase or no change in sFcεRI in the sample after the therapy relative to the sample before the therapy indicates that the subject is not responsive to therapy.

In certain embodiments, the method further comprises repeating steps (ii) and (iii) so as to monitor the efficacy of the therapy.

In certain embodiments, the IgE-mediated disorder may include but is not limited to:

allergy (such as asthma, atopic dermatitis, allergic rhinitis), esophagitis (e.g., eosinophilic esophagitis or EoE), gastroenteritis (e.g., eosinophilic gastroenteritis or EoG), hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis or necrotizing enterocolitis.

In some embodiments, the subject may be a human subject or a non-human subject. In some embodiments, the subject has a normal level of serum IgE.

The sample may be blood, serum, plasma, lymph, saliva or urine.

In yet a further aspect, the present disclosure provides a method of evaluating responsiveness to an immunotherapy in a subject. The method comprises: (i) detecting or measuring a level of sFcεRI in a sample from a subject in need of an immunotherapy collected before the immunotherapy; (ii) detecting or measuring a level of sFcεRI in a sample collected from the subject after the immunotherapy; and, (iii) comparing sFcεRI levels in the samples collected before and after the immunotherapy, wherein an increase in the level of sFcεRI in the sample collected after the immunotherapy relative to the sample collected before the immunotherapy indicates that the subject is responsive to the immunotherapy.

In some embodiments, a decrease or no change in the level of sFcεRI in the sample collected after the immunotherapy relative to the sample collected before the immunotherapy indicates that the subject is not responsive to the immunotherapy.

In some embodiments, the subject has a cancer. In some embodiments, the immunotherapy is a cancer immunotherapy.

The sample may be blood, serum, plasma, lymph, saliva or urine.

In another aspect, the present disclosure provides a method of evaluating responsiveness to an immunotherapy in a subject. The method comprising: (i) measuring a level of sFcεRI in a sample from a subject in need of an immunotherapy collected before the immunotherapy, (ii) measuring a level of sFcεRI in a biological sample collected from the subject after the immunotherapy, (iii) comparing sFcεRI levels in the samples collected before and after the therapy, wherein a decrease in the level of sFcεRI in the sample collected after the immunotherapy relative to the sample collected before the immunotherapy indicates that the subject is responsive to the immunotherapy.

In some embodiments, an increase or no change in the level of sFcεRI in the sample collected after the immunotherapy relative to the sample collected before the immunotherapy indicates that the subject is not responsive to the immunotherapy.

In some embodiments the subject has an allergic disease. In some embodiments, the immunotherapy is an allergy immunotherapy.

Another aspect of the disclosure is directed to a method of treating an IgE-mediated disorder in a subject. The method comprises administering a composition comprising sFcεRI to a subject having or at risk of having an IgE-mediated disorder in an amount effective to treat the disorder. In some embodiments, the composition further comprises a pharmaceutically acceptable carrier.

In certain embodiments, the IgE-mediated disorder may include but is not limited to: allergy, esophagitis (e.g., eosinophilic esophagitis or EoE), gastroenteritis (e.g., eosinophilic gastroenteritis or EoG), hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis or necrotizing enterocolitis.

In some embodiments, the subject may be a human subject or a non-human subject. In some embodiments, the subject has a normal level of serum IgE.

The sample may be blood, serum, plasma, lymph, saliva or urine.

In a further aspect, the disclosure provides an assay for detecting sFcεRI in a sample. The assay comprises an agent that binds to sFcεRI and a solid substrate. The agent may be immobilized on the solid substrate, and the sFcεRI may be detected with a probe.

In some embodiments, the agent is a recombinant IgE. The binding of the agent to sFcεRI may be conformation-specific or not conformation-specific.

In some embodiments, the assay is an enzyme-linked immunosorbent assay (ELISA).

The sample may be blood, serum, plasma, lymph, saliva or urine.

### BRIEF DESCRIPTION OF THE DRAWINGS

FIG. 1 provides a panel of six immunohistochemical images taken from biopsies, showing that FcεRI is the main IgE-binding component on intraepithelial inflammatory cells infiltrating the esophageal epithelium of eosinophilic esophagitis subjects. Left panel shows esophageal biopsies from patients with eosinophilic esophagitis the right panel shows positive control sections from tonsils. Immunohistochemistry with an anti-FcεRI specific mAb (upper panel), anti-CD23 specific mAb (middle panel) and an anti-galectin-3 specific mAb (lower panel).

FIG. 2 is a schematic illustration of the tetrameric and trimeric forms of the high affinity IgE receptor, FcεRI. The high affinity IgE receptor, FcεRI is a multimeric immune recognition receptors formed by the ligand-binding α-chain, one β-chain and a pair of disulfide-linked γ-subunits. In humans, FcεRI is expressed as a tetrameric receptor on the surface of basophils and mast cells. In the absence of β-transcripts, humans express a trimeric version of FcεRI on eosinophils. Monocytes, macrophages and professional antigen presenting cells, like dendritic cells and Langerhans cells.

FIG. 3 provides a schematic illustration of IgE-FcεRI-mediated activation of cells of the immune system. Human FcεRI is expressed on mast cells and basophils in its tretrameric isoform (tetramer: FcεRI αβγ2, left panel). Eosinophils, dendritic cells and macrophages express the trimeric isoform of the receptor (trimer: FcεRI αβγ2, right panel). Allergens activate immune cells by crosslinking FcεRI complexes that are loaded with specific IgE. The IgE-binding α-chain associates with the signaling subunits, β and γ. Phosphotyrosin-based signals via ITAMs of the signaling units induce degranulation of mast cells, basophils and eosinophils. Dendritic cells and macrophages internalize allergen-IgE complexes after FcεRI crosslinking and shuttle them towards antigen presentation to elicit T helper cell responses.

FIG. 4 provides a schematic and a panel of two fluorescent images of cultured transfected MelJuso cells, demonstrating internalization of FcεRI. FIG. 4A. Scheme for FcεRI activation with mAbs. FIG. 4B. Crosslinking of FcεRI at the surface of αγMelJuso induces receptor internalization. αγMelJuso were incubated with mAb CRA-1 for 20 min. Cross-linking of surface receptors that reacted with mAb CRA-1 was performed with a goat-anti-mouse conjugated Alexa-568. This condition was defined as time point zero (0 min, left picture). Activated FcεRI forms clusters as evident by the punctuate staining at the cell surface. After further 40 min of incubation at 37° C., the surface engaged receptors are found inside the cell (right picture).

FIG. 5 provides a schematic and a panel of two fluorescent images of OVA, demonstrating FcεRI-mediated antigen uptake and degradation in αDC2.4 as a model for antigen presenting cells. FIG. 5A. Model of receptor specific uptake: cells are incubated with NP-specific cIgE over night to engage surface expressed receptors monovalently. Cells are chilled to block phagocytotic uptake of antigen. Haptenized OVA (OVA-NP) is used as a model antigen and loaded on cells in the cold. Excess antigen is washed away in the cold and cells are moved to 37° C. for further analysis. FIG. 5B. Allergen-IgE mediated crosslinking of FcεRI on the surface of αDC2.4 results in antigen internalization and degradation. Receptor loading was performed at 4° C. (left picture). After the cells are moved to 37° C., the receptor internalized and the antigen is degraded (right picture).

FIG. 6 illustrates a sandwich ELISA for the detection of a soluble form of FcεRI. The alpha-chain specific mAb Cra 1 is used as a coating reagent. After a 4 h binding step, sFcεR is detected with human IgE and a peroxidase-coupled anti-human IgE-reagent.

FIG. 7 provides two graphs showing quantification of sFcεRI in the supernatant of MelJuso cells. FIG. 7A. Kinetics of sFcεRI release into culture supernatants. Supernatants were harvested 4, 8, 24 and 32 h after receptor activation. ELISA measurements showed an accumulation of sFcεRI over time. FIG. 7B. As a control we show that the parental MelJuso does not produce sFcεRI.

FIG. 8 provides a dilution curve showing sFcεRI is detected in patient serum.

FIG. 9 provides an image of immunoblot following IgE immunoprecipitation of sFcεRI from supernatant of activated MelJusoαγ. FIG. 9A. Immunoprecipitation of sFcεRI from supernatant collected 36 h after receptor activation is compared to alpha-chain in cell lysates. The alpha-chain was detected by immunoblotting with mAb 19-1. FIG. 9B. IgE precipitation cleared sFcεRI from the cell culture supernatant. Levels of sFcεRI in the supernatant were checked pre- and post-IP by ELISA.

FIG. 10 provides a bar graph showing that the production of sFcεRI is modulated by IFN-γ. Compare the black bar left of the dotted line (minus IFNγ) with the black bar on the right side of the dotted line (plus IFNγ).

FIG. 11 is a schematic illustration of a screening scheme for biomarkers using the Nanostring technology.

FIG. 12 illustrates experimental design to show that FcεRI activation induces the production of a soluble form from stable cell lines. FIG. 12A. Scheme of receptor activation. FIG. 12B. IgE precipitation of sFcεRI from supernatant of activated MelJusoαγ. Immunoprecipitation of sFcεRI from supernatant collected 36 h after receptor activation is compared to alpha-chain in cell lysates. FcεRI alpha was detected with mAb 19-1. FIG. 12C. Scheme of ELISA for the detection of sFcεR. FIG. 12D. IgE precipitation cleared sFcεRI from the cell culture supernatant. Levels of sFcεRI in the supernatant were checked pre- and post-IP by ELISA.

FIG. 13 provides an immunoblot image showing that FcεRI immunoreactivity is detected in both the soluble fraction (supernatant) and the exosome fraction. IgE immuno-precipitation from exosome depleted supernatant (lane 1) and from the exosome fraction (lane 2). The sFcεRI is detected by immuno-blotting with mAb19-1.

FIG. 14 illustrates ELISA models for detecting and measuring IgE-binding protein. FIG. 14A. Model for detection from cell lysates. cIgE anti-NP was coupled to a NIP-OVA precoated plate. After ON blocking, plates were reacted with serial dilutions of NP-40 cell lysates of HeLaαγ or untransfected HeLa cells at the indicated concentrations. Binding of FcεRI to its natural ligand IgE was detected with biotinylated mAB Cra-1 and Streptavidine HRP. FIG. 14B. Comparison of optical density (OD) measure at 450 nm from serial dilutions of cell lysates. FIG. 14C. Comparison of two different concentrations of the detection mAb.

FIG. 15 provides schematic representations of modified anti-alpha ELISA. FIG. 15A. Elisa for the detection of human FcεRI independent of its folding or IgE-loading stage. FIG. 15B. Detection of human IgE with the Hela-alpha lysates. FIG. 15C. Detection of human IgG-autoantibodies with an ELISA based on Hela-alpha lysates.

FIG. 16 provides experimental illustration and human data from ELISA. FIG. 16A. Schematic of the alpha chain-specific ELISA. FIG. 16B. Distribution of total FcεRI levels in 122 pediatric patients. FIG. 16C. Serum levels of sFcεRI correlate with serum IgE levels in atopic population (left); No correlation was found in non-atopic patients (right). FIG. 16D. Some individuals have high levels of sFcεRI but low/normal levels of IgE. FIG. 16E. sFcεRI circulates as a free or an IgE-complexed protein in human serum. By omitting the IgE-loading step in the ELISA protocol, circulating complexes of IgE and sFcεRI were measured. The fraction of free sFcεRI was then calculated as OD(total sFcεRI)−OD(IgE-sFcεRI complexes)=OD free sFcεRI (graph displays n=14 patients). OD, optical density (450 nm).

FIG. 17 demonstrates that a soluble form of the high affinity IgE receptor, FcεRI, is found in human serum. FIG. 17A. Soluble FcεRI (sFcεRI) was precipitated from serum with IgE-loaded NIP-beads and eluted with non-reducing Laemmli sample buffer. Eluates were separated on 12% non-reducing SDS-PAGE gels, transferred to PVDF membranes and probed with anti-FcεRI-alpha mAb 19-1 followed by peroxidase (HRP)-conjugated goat-anti-mouse IgG for detection of precipitated α-chain (left upper blot). As a positive control, cell surface FcεRI from MelJusoαγ cells was precipitated from cell lysates (right upper blot). In the low molecular weight range, blots were probed with an anti-FcεRI-gamma polyclonal serum. sFcεRI does not associate with the gamma chain (lower panel). FIG. 17B. Representative blot of immunoprecipitations from negative (first lane, 17A) and positive (right lane, 17B) serum specimens.

FIG. 18 demonstrates that sFcεRI blocks IgE loading of cell surface-expressed FcεRI. FIG. 18A. MelJusoαγ were loaded for 30 min on ice with a 1:2 mix of cIgE and PBS-buffer (left panel) versus cIgE and sFcεRI-positive serum (right panel) FIG. 18B. Serial dilution of cIgE with cell-culture derived sFcεRI (black squares) versus cIgE diluted with medium control (open circles). Cell-bound cIgE was stained with PE-conjugated hapten and measured by flow cytometry. Serum-sFcεRI prevents cIgE binding to cellular FcεRI. Define MFI here for panel B's y-axis.

### DETAILED DESCRIPTION OF THE DISCLOSURE

The inventors of the present application discovered that a soluble form of FcεRI is generated in vivo under certain conditions. The present disclosure is based, at least in part, on the discovery that a soluble form of the alpha chain of FcεRI (sFcεRI) is detected in samples collected from subjects with eosinophilic esophagitis (EoE), an IgE-mediated disorder. Based on this finding, the present disclosure provides a novel biomarker molecule for a variety of IgE-mediated disorders and thus provides an alternative diagnostic tool for these disorders.

For the other known Fc receptors, excluding FcεRI, soluble forms of the alpha chains had been described in the literature. A prevailing view is that soluble alpha chains of Fc receptors generally may function as in vivo modulators of Ig-mediated activation of the immune system. In this respect, sFc receptors provide a link between the humoral and the cellular arm of the immune system.

FcεRI is an activating immune receptor of the immunoglobulin superfamily, like other Fc receptors such as CD32, CD16, CD64 or CD89. The structural characteristics and mode of cell activation described above for FcεRI are actually a unifying principle for all of these Fc receptors. The alpha chains carry the ligand-binding domain of the protein complex. Via their transmembrane regions, these alpha chains associate with subunits which use ITAM-based activation of kinases to signal immune activation after Ig-mediated receptor crosslinking.

As described herein, the inventors of the present application discovered that IgE-mediated cell activation induces the release of a soluble form of the FcεRI alpha chain (s FcεRI). This sFcεRI is found in a free form, as well as complexed with its ligand IgE in human serum. Due to the high affinity of ligand interaction characteristic for this IgE-receptor, sFcεRI is likely to act as a potent regulator of IgE responses in vivo.

The term “FcεRI” as used herein is intended to encompass multiple forms of the protein complex, including the trimeric form and the tetrameric form, as described herein. In some embodiments, FcεRI is a trimeric complex comprised of an α (alpha) chain and two γ (gamma) chains. In other embodiments, FcεRI is a tetrameric complex comprised of an α (alpha) chain (GenBank Accession No. NP_{—}001992.1), a β (beta) chain and two γ (gamma) chains. The term “FcεRI” also includes fragments of the receptor complex. In the context of the present disclosure, the term is used to refer to a full-length as well as fragments of FcεRI that contain at least one binding site for IgE defined by crystallography as the two immunoglobuline-like loops in the extracellular domain of the alpha chain (Crystal structure of the human high-affinity IgE receptor. Garman S C, Kinet J P, Jardetzky T S. Cell. 1998 Dec. 23; 95(7):951-61; and Structure of the Fc fragment of human IgE bound to its high-affinity receptor Fc epsilonRI alpha. Garman S C, Wurzburg B A, Tarchevskaya S S, Kinet J P, Jardetzky T S. Nature. 2000 Jul. 20; 406(6793):259-66.).

The term “soluble FcεRI” or “sFcεRI” refers to at least a partial fragment of the α chain of the FcεRI receptor complex and contains at least a binding site for IgE. Generally, sFcεRI corresponds to an extracellular portion of the FcεRI alpha chain. In some embodiments, sFcεRI corresponds to amino acids 26-201 of NP_{—}001992.1 (GenBank Accession No.). As described herein, it has been discovered that an alpha fragment is released (or shedded) into the blood stream in a subject with an IgE-mediated disorder, and can be used as a biomarker to diagnose such a disorder or to identify a subject having or at risk of having (e.g., predisposed of developing) such a disorder.

The terms “disorder” “disease” “condition” may be used interchangeably. For example, “an allergic disorder” and “an allergic condition” are not intended to be distinct.

As discussed in more detail herein, two independent approaches have been taken to define new biomarkers for IgE-mediated disorders. First, a targeted approach was taken to evaluate the potential of a novel soluble form of FcεRI as a marker for diagnosis and monitoring of intervention strategies. This protein has been identified as a candidate based on the data, which provide evidence that this IgE-receptor is the main IgE binding structure in the esophagus of EoE subjects. Second, assays useful for a broad screening of markers which are indicative of IgE-related conditions is contemplated that depend on FcεRI-mediated activation of the immune system. Such proteins make good candidates for predictive markers for chronicity and long-term complications of IgE-mediated disorders.

As used herein, the term “IgE-mediated disorders” includes allergic disorders, which are characterized by a general inherited propensity to respond immunologically to many common naturally occurring inhaled and ingested antigens and the continual production of IgE antibodies. Non-limiting examples of such disorders include allergic asthma, esophagitis (e.g., eosinophilic esophagitis or EoE), gastroenteritis (e.g., eosinophilic gastroenteritis or EoG), allergic rhinitis (ocular allergy, conjunctivitis), atopic dermatitis, food allergy, anaphylaxis, contact dermatitis, allergic gastroenteropathy, allergic bronchopulmonary aspergillosis and allergic purpura (Henoch-Schönlein). Atopic subjects often have multiple allergies, meaning that they have IgE antibodies to, and symptoms from, many environmental allergens, including seasonal, perennial and occupational allergens. Examples of seasonal allergens include pollens (e.g., grass, tree, rye, timothy, ragweed), while example perennial allergens include fungi (e.g., molds, mold spores), feathers, animal (e.g., pet or other animal dander) and insect (e.g., dust mite) debris. Examples of occupational allergens also include animal (e.g. mice) and plant antigens as well as drugs, detergents, metals and immunoenhancers such as isocyanates. Non-antigen specific stimuli that can result in an IgE-mediated reaction include infection, irritants such as smoke, combustion fumes, diesel exhaust particles and sulphur dioxide, exercise, cold or emotional stress. Specific hypersensitivity reactions in atopic and non-atopic individuals with a certain genetic background may result from exposure to proteins in foods (e.g., legumes, peanuts), venom (e.g., insect, snake), vaccines, hormones, antiserum, enzymes, latex, antibiotics, muscle relaxants, vitamins, cytotoxins, opiates, or polysaccharides such as dextrin, iron dextran or polygeline.

Other disorders associated with elevated IgE levels, that appear to be IgE-mediated and are relevant to the present disclosure include, but are not limited to: allergic bronchopulmonary mycoses, ataxia-telangiectasia, Churg-Strauss Syndrome, eczema, enteritis, gastroenteropathy, eosinophilic esophagitis, eosinophilic gastroenteritis, graft-versus-host reaction, hyper-IgE (Job's) syndrome, hypersensitivity (e.g., anaphylactic hypersensitivity, candidiasis, vasculitis), IgE myeloma, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis, parasitic diseases (e.g., trypanosomiasis), hypersensitivity vasculitis, Vernal keratoconjunctivitis (VKC), urticaria, Wiskott-Aldrich syndrome, glioma, ovarian cancer or leukemia.

Additionally, disorders that may be treatable by lowering IgE levels, regardless of whether the disorders themselves are associated with elevated IgE, and thus should be considered within the scope of “IgE-mediated disorder” include, but are not limited to: Addison's disease (chronic adrenocortical insufficiency), alopecia, hereditary angioedema, anigioedema (Bannister's disease, angioneurotic edema), ankylosing spondylitis, aplastic anemia, arteritis, amyloidosis, immune disorders, such as autoimmune hemolytic anemia, autoimmune oophoritis, autoimmune orchitis, autoimmune polyendocrine failure, autoimmune hemolytic anemia, autoimmunocytopenia, autoimmune glomerulonephritis, Behcet's disease, bronchitis, Buerger's disease, bullous pemphigoid, Caplan's syndrome (rheumatoid pneumoconiosis), carditis, celiac sprue, Chediak-Higashi syndrome, chronic obstructive lung Disease (COPD), Cogan-Reese syndrome (iridocorneal endothelial syndrome), CREST syndrome, dermatitis herpetiformis (Duhring's disease), diabetes mellitus, eosinophilic fasciitis, eosinophilic nephritis, episcleritis, extrinsic allergic alveolitis, familial paroxysmal polyserositis, Felty's syndrome, fibrosing alveolitis, glomerulonephritis, Goodpasture's syndrome, granulocytopenia, granuloma, granulomatosis, granuloma myositis, Graves' disease, Guillain-Barre syndrome (polyneuritis), Hashimoto's thyroiditis (lymphadenoid goiter), hemochromatosis, histocytosis, hypereosinophilic syndrome, irritable bowel syndrome, juvenile arthritis, keratitis, leprosy, lupus erythematosus, Lyell's disease, Lyme disease, mixed connective tissue disease, mononeuritis, mononeuritis multiplex, Muckle-Wells syndrome, mucocutaneous lymphoid syndrome (Kawasaki's disease), multicentric reticulohistiocystosis, multiple sclerosis, myasthenia gravis, mycosis fungoides, panninculitis, pemphigoid, pemphigus, pericarditis, polyneuritis, polyarteritis nodoas, psoriasis, psoriatic arthritis, pulmonary arthritis, pulmonary adenomatosis, pulmonary fibrosis, relapsing polychondritis, rheumatic fever, rheumatoid arthritis, rhinosinusitis (sinusitis), sarcoidosis, scleritis, sclerosing cholangitis, serum sickness, Sezary syndrome, Sjögren's syndrome, Stevens-Johnson syndrome, systemic mastocytosis, transplant rejection, thrombocytopenic purpura, thymic alymphoplasia, uveitis, vitiligo, or Wegener's granulomatosis.

The present disclosure identifies sFcεRI as a marker for IgE-mediated activation of the immune system—a step beyond conventional allergy diagnosis. Based on the fact that IgE regulates surface expression of FcεRI in allergic subjects, one might argue that this IgE receptor is an unlikely target structure for a biomarker for IgE-mediated conditions in subjects, such as those with EoE. If serum IgE levels and FcεR expression on peripheral blood cells indeed correlated, there would only be need for developing a more complete or sensitive form of measuring serum IgE. However, data indicate that this is not the case. Subjects with low serum IgE can have IgE that is bound to peripheral blood cells (unpublished observation). In these cases, subjects can carry substantial amounts of IgE at the cell surface of basophils and dendritic cells without presenting with elevated levels of IgE in the serum. It was also found that FcεRI is the main IgE-binding receptor on these cells. Taken together, these results suggest that cells in the peripheral blood bind IgE from the serum and indicate that this could be a mechanism to deplete IgE from the serum. Once immune activation occurs, these cells could drag their surface bound IgE to the site of inflammation, such as the esophagus in case of EoE subjects. Thus, cell surface bound IgE might be a better predictive marker than serum IgE for the development of EoE, and for allergy (IgE-mediated disorders) more generally. Thus, data described herein argue against the oversimplified notion that FcεRI expression directly correlates with serum IgE levels.

A general problem with the precise diagnosis of allergy is that a considerable proportion of patients exhibit allergic symptoms in the absence of elevated serum IgE, the current standard for diagnosis. (Ownby D R, Allergy testing: in vivo versus in vitro. Pediatr Clin North Am 1988; 35: 995-1009; and Novak N, Bieber T, Allergic and nonallergic forms of atopic diseases. J Allergy Clin Immunol 2003; 112: 252-262). One example for this is EoE, an allergic condition of the upper gastrointestinal tract. There is no currently clear consensus as to whether “non-allergic” (non-IgE mediated) EoE subjects exist, and if so, what would be a proper way to screen the subpopulation of subjects as these patients have now elevated serum IgE levels. Depending on the source of literature, IgE-mediated mechanisms play a role in up to 80% of EoE subjects. While this mechanism is certainly the dominant mechanism in most EoE subjects, a more complex pathophysiology is likely to underlie the disease. A substantial fraction of EoE subjects show no clinical presentations of allergy, e.g., no measurable serum IgE, have negative skin prick tests and no symptoms timely related to allergen exposure. Still, many of these subjects without elevated specific serum IgE respond positively to elimination of certain foods or an elemental diet. In these subjects, T-cell mediated mechanisms have been suggested by several studies. This idea is consistent with the notion that EoE may represent a delayed-type food hypersensitivity. It is possible that in the absence of elevated serum IgE cell-bound IgE in resident immune cells in the esophagus are activated by allergens and the immune system reacts with the production of sFcεRI. In this case, the EoE symptoms would still be dependent on an IgE-mediated mechanism and involve FcεRI activation, and therefore the strategy to use sFcεRI (which relies on the activation of FcεRI to be produced) as a biomarker as described here captures these subjects. This scenario can be envisioned for all patients that show allergic symptoms in the absence of elevated serum IgE (Novak N, Bieber T, Allergic and nonallergic forms of atopic diseases. J Allergy Clin Immunol 2003; 112: 252-262).

In some cases, a subject with an IgE-mediated disorder has one or more clinical symptoms, e.g., allergic symptoms. In other cases, a subject with an IgE-mediated disorder does not present significant clinical symptoms, e.g., the subject is asymptomatic. Without intending to be bound by any particular theory, a possible explanation for this discrepancy may be that sFcεRI protein may act as a buffer to sequester free serum IgE, preventing full manifestation of clinical symptoms during early stages of pathogenesis. Nevertheless, it is believed that the asymptomatic subject still has predisposition for, or at risk of, developing clinically symptomatic allergic disorders, presumably when serum IgE is elevated relative to available sFcεRI. Clinical symptoms of IgE-medicated disorders are known to one of ordinary skill in the art. In some embodiments, the subject has normal serum IgE level and has not developed clinical symptoms of allergy.

In some embodiments, a subject having or at risk of having an IgE-mediated disorder, and with or without clinical symptoms, may have an increased level of serum IgE. In some embodiments, a subject having or at risk of having an IgE-mediated disorder, and with or without clinical symptoms, has a normal level of serum IgE. Surprisingly, in any of these sub-populations of subjects with an IgE-mediated disorder, sFcεRI is detectable in the serum of the subject, or in another biological sample.

Normal total serum IgE levels may depend on the age group into which a subject falls. In some embodiments, normal total serum IgE levels are defined as follows: <10 IU/ml for ages 0-3 years, <25 IU/ml for ages 3-4 years, <50 IU/ml for ages 4-7 years, <100 IU/ml for ages 7-14 years, and <150 IU/mL for subjects older than age 14 as determined by a solid-phase ELISA (DiaMed Eurogen, Turnhout, Belgium) (Relationships between levels of serum IgE, cell-bound IgE, and IgE-receptors on peripheral blood cells in a pediatric population. (Dehlink E, Baker A H, Yen E, Nurko S, Fiebiger E. PLoS One. 2010 Aug. 16; 5(8). pii: e12204). In some embodiments, normal IgE levels are about 0.05% of the IgG concentration.

Thus, according to one aspect of the disclosure, a method for diagnosing an IgE-mediated disorder in a subject is provided. The detection accordingly is based on soluble (e.g., secreted or released) FcεRI in a sample, i.e., sFcεRI. The method involves first detecting or measuring a level of sFcεRI in the sample from a subject, then comparing the level of sFcεRI in the sample to a predetermined value. If the level of sFcεRI in the sample is above the predetermined value, the subject is diagnosed with an IgE-mediated disorder. In some cases, the diagnosis is a confirmation of a previous or preliminary diagnosis. In some circumstances, the subject does not have elevated serum IgE, notwithstanding clinical presentation of allergic conditions. In these cases, an affirmative diagnosis of an IgE-mediated disorder based on sFcεRI is particularly useful in the determination of the optimal treatment regimen.

In a related aspect of the disclosure, the method of diagnosing an IgE-mediated disorder in a subject involves comparing the level of sFcεRI in the sample to a predetermined value. If the level of sFcεRI in the sample is above the predetermined value, the subject is diagnosed with an IgE-mediated disorder. In some cases, the diagnosis is a confirmation of a previous or preliminary diagnosis. In some circumstances, the subject does not have elevated serum IgE, notwithstanding clinical presentation of allergic conditions.

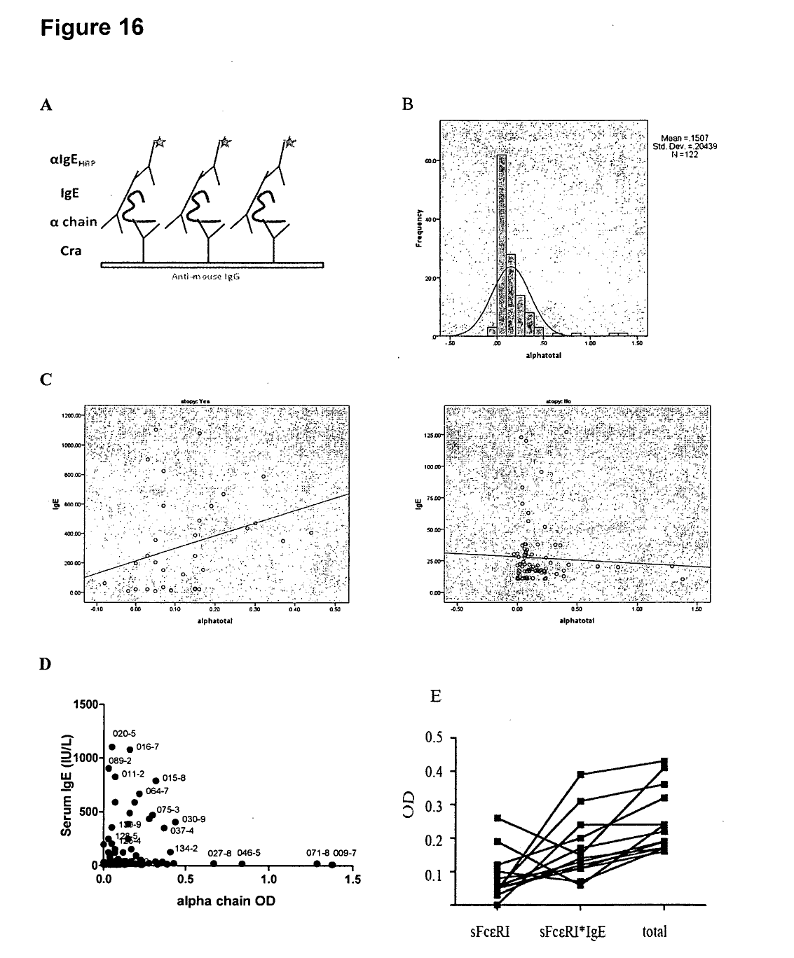

As used herein, “predetermined value” means a range of values that is determined statistically to be “the norm.” As an example, FIG. 16B provides a graph showing that in non-atopic pediatric population (n=122), the mean optical density (OD) measurement/readout for sFcεRI measured by the ELISA method described in the examples was determined to be 0.15 with a SD of 0.20. Accordingly, in some embodiments, the predetermined value is an OD of 0.20. In some embodiments, the predetermined value is an OD of 0.30. In some embodiments, the predetermined value is an OD of 0.40. In some embodiments, the predetermined value is an OD of 0.50. One of ordinary skill in the art can readily determine statistically sound range of values to be established as a predetermined value suitable for specific purposes.

The predetermined value can take a variety of forms. It can be a single cut-off value, such as a median or mean. It can be established based upon comparative groups, such as, for example, where the risk in one defined group is double the risk in another defined group. It can be a range, for example, where the tested population is divided equally (or unequally) into groups, such as a low-risk group, a medium-risk group and a high-risk group, or into quartiles, the lowest quartile being subjects with the highest risk and the highest quartile being subjects with the lowest risk, or into tertiles the lowest tertile being subjects with the highest risk and the highest tertile being subjects with the lowest risk. The predetermined value may be a cut-off value which is predetermined by the fact that a group having a sFcεRI level more than the cut-off value demonstrates a statistically significant increase in the risk of having an IgE-mediated disorder as compared to a comparative group. In some embodiments the comparative group is a group having a lower level of sFcεRI.

The predetermined value can depend upon the particular population of subjects selected. In some embodiments, the predetermined value depends on the age of the subjects selected. Accordingly, the predetermined values selected may take into account the category in which a subject falls. Appropriate ranges and categories can be selected with no more than routine experimentation by those of ordinary skill in the art. The predetermined value will depend, of course, upon the characteristics of the subject population in which the subject lies. In characterizing risk, numerous predetermined values can be established.

A level of sFcεRI above the predetermined value indicates the subject has or at risk of developing a clinically symptomatic IgE-mediated disorder. In some embodiments, the subject with a level of sFcεRI above the predetermined value presents clinical symptoms of allergic conditions. In some embodiments, the subject with a level of sFcεRI above the predetermined value is asymptomatic. In some embodiments, the predetermined value, as measured by ELISA followed by optic density readout, using the protocol described herein or equivalent thereof, is between 0.00 OD and 0.20 OD. In some embodiments, optic density readout is between 0.00 OD and 0.30 OD. In some embodiments, optic density readout is between 0.00 OD and 0.40 OD. In some embodiments, optic density readout is between 0.00 OD and 0.50 OD. Non-limiting embodiment is provided in Examples (see, for example, FIG. 16). In some embodiments, the predetermined value is at or near 0.15 OD, e.g., between about 0.00 OD and 0.30 OD. In some embodiments, the predetermined value is an OD of 0.2. In some embodiments, the predetermined value is an OD of 0.3. In some embodiments, the predetermined value is an OD of 0.4. In some embodiments, the predetermined value is an OD of 0.5. The predetermined value can depend upon the particular population of subjects selected. Accordingly, the predetermined values selected may take into account the category in which a subject falls.

The level of the sFcεRI for the subject can be obtained by any art recognized method. Typically, the level is determined by measuring the level of sFcεRI in a body fluid, for example, blood, serum, plasma, lymph, saliva, urine, and the like. The level can be determined by ELISA, or other immunoassays or other conventional techniques for determining the presence of sFcεRI. Conventional methods may include sending a sample(s) of a subject's body fluid to a commercial laboratory for measurement.

As used herein, a “subject” refers to an animal, preferably a mammal, which expresses at least one form of the FcεRI protein. In preferred embodiments, the subject is a human subject. In other embodiments, the subject is a non-human mammal, including but are not limited to, dog, cat, horse, etc.

Allergic individuals upregulate cell surface expression of FcεRI (Gould H J, Sutton B J, IgE in allergy and asthma today. Nat Rev Immunol 2008; 8: 205-217). Monomeric interaction with IgE traps the receptor on the cell surface. Allergen-specific crosslinking of these receptors is responsible for the complicated activation pattern that accounts for allergic symptoms. Activation of FcεRI plays a role in immediate-type allergic reactions, with histamine release from mast cells and basophils. The role of FcεRI activation for the delayed-type response is less well understood. The inventors of the present disclosure recently demonstrated that FcεRI-mediated antigen presentation shifts the immune response towards Th2 in a transgenic mouse that expresses FcεRI constitutively on dendritic cells (unpublished data).

Amidst increasing reports of incidences of IgE-associated disorders, certain types of conditions pose a particular challenge in the diagnosis and monitoring of the subjects' response to a therapy due to the invasive nature of procedures involved. For example, a number of IgE-mediated disorders are triggered by allergic reactions to food or environmental allergens and affect the gastrointestinal tissues of the subjects. These disorders include, eosinophil-associated gastrointestinal disorders, including eosinophilic esophagitis (EoE) and eosinophilic gastroenteritis (EoG), which represent a spectrum of increasingly recognized inflammatory diseases characterized by gastrointestinal symptoms and eosinophilic infiltration of the gastrointestinal tract (e.g., the esophagus), in the absence of parasitic infection, vasculitis, neoplasm or other known causes of eosinophilia.

EoE is an emerging, painful and sometimes devastating inflammatory disease of the esophagus, leading to swallowing problems, food refusal, pain, food intolerance, dysphagia and failure to thrive in otherwise healthy infants and children. In older children and adults it produces dysphagia and food impactions. Untreated, subjects with EoE develop complications such as esophageal strictures, long segment narrow caliber esophagus, esophageal dysmotility, and even cases of Boerhaave's have been reported. Food impactions requiring endoscopic removal are not uncommon. A four-fold increase in disease prevalence in children with EoE in the Midwest United States is described over a period from 2000 to 2003. In addition, an incidence of ˜1:10,000 children was reported. The increasing incidence of EoE is probably related to the recent upsurge in allergic diseases caused in part by our rapidly changing environment. Conclusive evidence shows that EoE is likely caused by allergy to food or other environmental antigens. EoE pathogenesis thus likely depends on IgE and IgE-receptors expressed in the esophageal tissue.

IgE-mediated allergic responses in EoE are mostly Type I hypersensitivity reactions and are associated with common food allergies, such as milk protein or peanut allergy. The same mechanism of IgE-mediated activation of the immune system is probably also responsible for diseases like allergic colitis and eosinophilic gastrointestinal disorders more generally. Allergic subjects have high serum IgE and high IgE-receptor levels on effector cells of the innate and adaptive immune system. Humans express three different IgE-receptors: CD23, galectin 3 and FcεRI. CD23, also known as FcεRII, is a low affinity IgE receptor and mainly used for IgE-trafficking on epithelial cells in the gastro intestinal tract. Galectin 3 is another low affinity IgE receptor, but with poorly defined functions for the gastrointestinal immune system. FcεRI is the high-affinity receptor for IgE. Human FcεRI is expressed on the surface of mast cells and basophils.

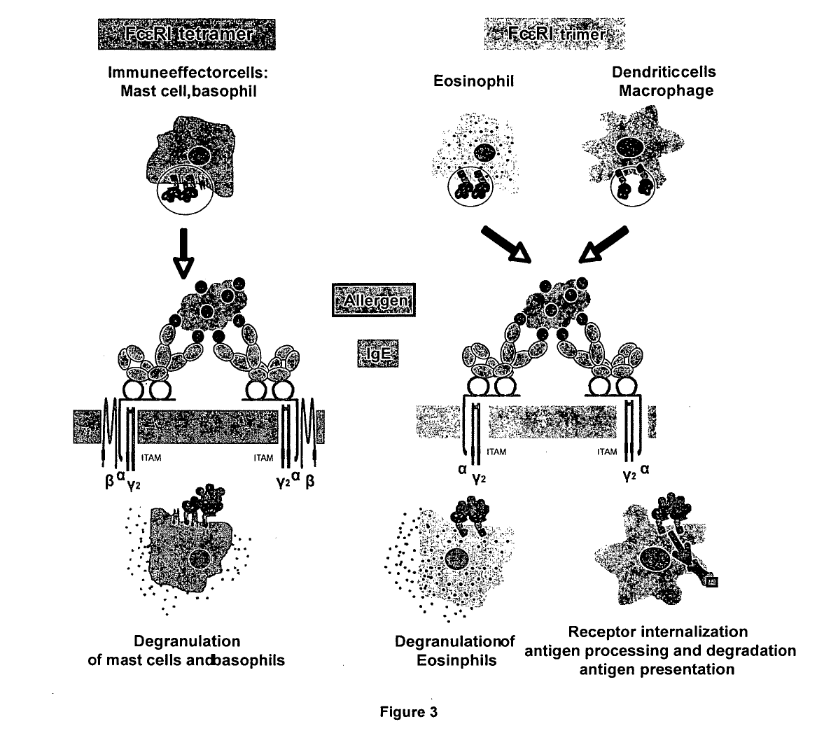

Studying asthma in αTG animals, it was discovered that the immune infiltrate in the lungs of αTG mice is typified by an influx of eosinophils when the dendritic cells can engage an IgE-mediated antigen presentation pathway. It is thus conceivable that IgE-mediated antigen presentation by Langerhans cells is critical for the influx of eosinophils into the esophageal tissue, which is a hallmark for EoE. A summary of the possible consequences of FcεRI-mediated activation of immune cells for EoE is shown in FIG. 3.

With respect to the field of EoE, major advances in the understanding of the pathophysiology have been accomplished. However, there has been a significant delay in the development of effective treatments. One of the main limitations has been the difficulty to assess subjects' responsiveness to therapy (e.g., effectiveness of the therapy) given the necessity to perform invasive procedures, such as gastrointestinal endoscopy and biopsy as the only reliable way for evaluation. The identification of sFcεRI as a biomarker for EoE and other related disorders undoubtedly changes the approach to the diagnosis and management of subjects with EoE as well as other related conditions that previously required invasive diagnostic and monitoring procedure, as it opens the possibility to establish new diagnosis and new and more effective treatment modalities in a non-invasive and cost-effective manner. Having a serum-based biomarker increases the effectiveness of the treatment, as it will allow more accurate and timely changes and adjustment in therapy. Being able to avoid repeated endoscopies will not only have a positive impact in the medical care of the subjects, but it will also reduce the possibility of complications related to the invasive nature of the procedure and the cost of taking care of the subjects. It is also useful for the monitoring of other IgE mediated conditions. Thus, the present disclosure may have broadly applicable impact on clinical practice and child health.

Based on these observations, EoE is an excellent disease model to examine a broader class of allergic disorders, namely, IgE-mediated disorders. As stated above, current treatment and evaluation of EoE are highly invasive, as an upper endoscopy is needed to monitor clinical progression and response to therapy, subjecting the infants and children with this disease to repeated upper gastrointestinal endoscopies. Another important aspect of EoE is that is has a profound impact on normal childhood development of feeding practices. One of the most fundamental aspects of child development and health in this age group has to do with feeding and the mother-child relationship formed over this activity. As such, the repercussions of painful swallowing (or even failure to swallow) caused by EoE extend way beyond the immediate aspects of nutrition, growth, and physical health.

As demonstrated in the Examples, the data provided herein support a role of FcεRI-mediated immune activation in the esophagus of subjects suffering from EoE. Based on the knowledge of the art, and as alluded to above, it is reasonably contemplated that the same mechanism of pathogenesis applies to other IgE-mediated disorders. Beyond the defined role of tetrameric FcεRI on mast cells and basophils in immediate type I allergic reactions, it is believed that expression of trimeric FcεRI on human eosinophils and antigen presenting cells is critically involved in the pathology of IgE-mediated disorders, such as EoE. An additional important argument is that anti-IgE therapy in subjects with eosinophilic gastroenteritis improves symptoms and down regulates FcεRI expression. We have collected a solid set of evidence that IgE-FcεRI-mediated activation of immune cells is critical in the pathology of EoE.

The urgent need to define novel biomarkers for IgE-mediated disorders such as EoE results from the current form of diagnosis of the disease. Because of a complete lack of alternatives, the diagnosis of EoE is performed histologically from esophageal biopsies obtained via upper endoscopy. The Gold Standard for the diagnosis of EoE is the presence of esophagitis with >15 eosinophils per high power field, unresponsive to at least 4 weeks of adequate dosages of proton pump inhibitors to block gastric acid secretion. Children with reflux esophagitis are commonly treated with proton pump inhibitors and will have esophageal biopsies characterized by basal zone hyperplasia, inflammatory cell infiltrate, and fewer than 15 eosinophils per high power field. Children with normal esophageal biopsies are categorized as normal based on the absence of infiltrating cells in the esophageal biopsies. Repeated endoscopy is mandatory to assure proper care of subjects suffering from EoE, but endoscopies are highly invasive, a burden on the subject, costly and rate limiting in discovering new approaches to treatment and prevention.

It has been observed that serum IgE does not always correlate with EoE. This is rather surprising in view of EoE being classified as a Th2 type allergy. Similarly, eotaxin levels are poor predictors of disease development despite the fact that esophageal tissue in subjects with EoE contains a marked increase in eosinophils. It has been shown that the EoE immune infiltrate is characterized by high numbers of FcεRI-receptor positive cells, like mast cells and dendritic cells. Human eosinophils are also known to express trimeric FcεRI. These observations led to the inventors of this application to discover that cell activation of the human trimeric FcεRI is critical and that analysis of this activation pathway is a new biomarkers for IgE-mediated disorders.

The present disclosure thus provides a non-invasive and cost-effective method for evaluating the effectiveness of a therapy for an IgE-mediated disorder in a subject. The method involves comparing a level of sFcεRI in a sample from the subject before and after the therapy and determining whether the subject is responsive to the therapy by monitoring sFcεRI levels. In some embodiments, the monitoring step may be started only after a therapy has already begun. Optionally, sFcεRI levels are monitored over a period of time. Duration of the period of time for monitoring will vary based on a number of factors, such as the nature of the IgE-mediated disorder, severity thereof, age of the affected subject, specific route of administration, etc. In any of these embodiments, the method according to this disclosure in some cases reduces or eliminates the need for invasive and costly medical procedures for assessing the efficacy of treatment.

In some embodiments, the subject may have been undergoing the therapy for at least 1, 2, 3, 4, 5, 6, 7 days or more. In some embodiments, the subject may have been undergoing the therapy for at least 1, 2, 3, 4, 5, 6, 7, 8, 9, 10, 11, 12 weeks or more. In some embodiments, the subject may have been undergoing the therapy for at least 3, 4, 5, 6, 7, 8, 9, 10, 11, 12 months or more.

The present disclosure thus offers useful applications for personalized medicine directed to IgE-mediated disorders. For example, embodiments of the present disclosure can be used to evaluate the effectiveness of a certain treatment and examine a subject's responsiveness to the therapy. Because of its non-invasive nature, the evaluation procedures may be repeated to monitor the subject's responsiveness and progress over time in a reliable, cost-effective manner. Therefore, the present disclosure contributes to the development of a new and improved set of intervention strategies.

The instant disclosure also provides a useful method for evaluating responsiveness to an immunotherapy in a subject who is receiving such therapy. The method involves the detection (e.g., measurement) of sFcεRI in a sample from a subject who receives an immunotherapy. Typically, monitoring of serum sFcεRI levels is carried out before, during and after the applicable immunotherapy. When an increased level of serum sFcεRI is detected in the sample after the subject receives the immunotherapy, or after the treatment regimen has begun, then it is indicative of the subject's responsiveness to the immunotherapy.

A subject in need of immunotherapy includes a subject who will benefit from boosting of an immune response. In some embodiments, the subject has cancer. In some embodiments, the subject has cancer and undergoes cancer immunotherapy. Thus, this method for evaluating the responsiveness of the subject to an immunotherapy is useful as part of managing cancer treatment regimen, because often it takes much longer time before other parameters become measurable, such as the size of a tumor, in response to an immunotherapy. But using this method described herein, the subject and his or her physician can confirm that the subject is responding to the therapy before clinical effects can be measured. Based on this evaluation, the physician may continue with the immunotherapy, if there is favorable immune response, or discontinue or change to another therapy if no significant immune response is triggered by the immunotherapy.

This aspect of the disclosure is particularly relevant in the context of cancer immunotherapy but is not limited thereto. Cancer immunotherapies are well known in the art and include, without limitation, monoclonal antibody therapy and radioimmunotherapy. Non-limiting examples of monoclonal antibodies used as cancer immunotherapy include: Alemtuzumab, Bevacizumab, Cetuximab, Gemtuzumab ozogamicin, Rituximab and Trastuzumab. On the other hand, radioimmunotherapy involves the use of radioactively conjugated antibodies against cellular antigens (e.g., tumor antigens) and include, without limitation, Ibritumomab tiuxetan and Tositumomab. Any immunotherapy is applicable to the instant disclosure, so long as the therapy, when properly exerting its effect, triggers IgE stimulation in the subject.

Also described herein is a novel form of a soluble IgE receptor, sFcεRI, with a potential to modulate IgE-mediated immune events in vivo. This is a novel serum modulator and of high interest with regards to our understanding of allergy more generally. As described, FcεRI is a high affinity IgE binding protein. Therefore, the soluble form of the FcεRI protein (sFcεRI) described in this disclosure may be useful as a therapeutic for treating conditions that are caused by an elevated level of IgE. High levels of serum IgE is a common observation seen in allergic subjects. It is believed that suppressing serum IgE in these conditions is beneficial for the treatment of allergic conditions. In fact, a number of therapeutics has been developed, which work by targeting IgE, such as human anti-IgE antibody therapy. Effectiveness of such strategy has been established. Therefore, the soluble form of FcεRI (sFcεRI) described herein should also be useful to block the action of circulating IgE, which causes hypersensitivity in allergic subjects. To this end, a recombinant sFcεRI fragment containing a high affinity binding site for serum IgE should be effective in sequestering excess IgE in vivo. The criteria for the target population for such a therapy may be readily established by measuring serum IgE levels in the subjects. Once an elevated IgE level is confirmed, the subject can benefit from administration of a pharmaceutical composition comprising sFcεRI. Such compositions are embraced by this disclosure.

The term “treat” or “treatment” is intended to include prophylaxis, amelioration, prevention or cure from the condition.

The composition as described above may further comprise a pharmaceutically acceptable carrier. The art is familiar with a variety of pharmaceutically acceptable carriers which are suitable for use in formulating a composition. A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Various modes of administration, which are well known in the art, are contemplated and thus are embraced by this disclosure. For example, sFcεRI may be administered orally, sublingually, buccally, intranasally, intravenously, intramuscularly, intrathecally, intraperitoneally, or subcutaneously. In some embodiments, the modes of administration for sFcεRI include but are not limited to intravenous injection and nasal spray.

The subject who may benefit from receiving such treatment is limited by the clinical presentation of elevated IgE, e.g., high serum IgE. Some allergic subjects, who exhibit clinical allergic symptoms, but do not have high IgE, are excluded from a preferred target population. In the context of instant disclosure, such composition is suitable for use for human subjects as well as non-human subjects, such as for veterinary use. It is well documented in the relevant literature that many non-human animals, including dogs, cats and horses, develop IgE-mediated allergic conditions. As such, irrespective of specific allergens to which a particular subject is hypersensitive, the composition comprising sFcεRI should be effective in buffering the IgE effects in vivo. Thus, the method of treating an IgE-mediated disorder as described herein provides a cost-effective alternative to anti-IgE-based therapeutics.

The therapeutic sFcεRI may be administered alone, in a pharmaceutical composition or combined with other therapeutic regimens. sFcεRI and optionally other therapeutic agent(s) may be administered simultaneously or sequentially. When the other therapeutic agents are administered simultaneously they can be administered in the same or separate formulations, but are administered at the same time. The other therapeutic agents may be administered sequentially with one another and with sFcεRI when the administration of the other therapeutic agents and the sFcεRI is temporally separated. The separation in time between the administration of these compounds may be a matter of minutes or it may be longer.

Subjects receiving such treatment may be monitored based on clinical symptoms and/or by the use of conventional IgE tests. As used herein, “an amount effective to treat” the disorder is an amount of the therapeutic that results in a reduced level of serum IgE in the subject when administered. Effective treatment regimen may be determined by monitoring changes in IgE levels before and after the subject receives the therapy. Accordingly, suitable dose, (e.g., frequency of therapy, duration, etc.) may be established for the particular individual.

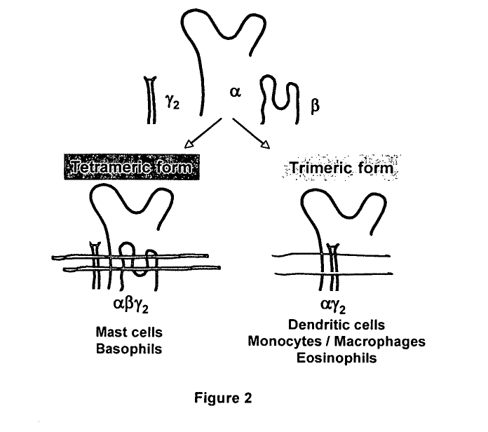

Evidence indicates that FcεRI is one of the key molecules in the pathophysiology of all allergic reactions, i.e., IgE-mediated disorders. FcεRI shares with other Fc receptors the overall structure of a ligand-binding immunoglobulin domain-containing protein associated with signaling subunits that regulate cellular activation. A receptor model is shown in FIG. 2. In rodents, FcεRI is exclusively expressed as a tetrameric receptor composed of the ligand-binding α-chain, one β-chain and a pair of disulphide-linked γ-subunits on the surface of basophils and mast cells. In the absence of β-transcripts, humans can express a trimeric version of FcεRI on eosinophils and antigen presenting cells, such as dendritic cells and Langerhans cells. The difference in FcεRI expression between murine and human cells might be one reason why it is challenging to establish reliable murine models for EoE. Murine models would not express the same type of IgE-binding structure as FcεRI on Langerhans cells, which is the main IgE-binding structure in the esophagus of EoE subjects. The immunologic relevance of the trimeric FcεRI receptor is likely not only under-investigated, but also underrated.

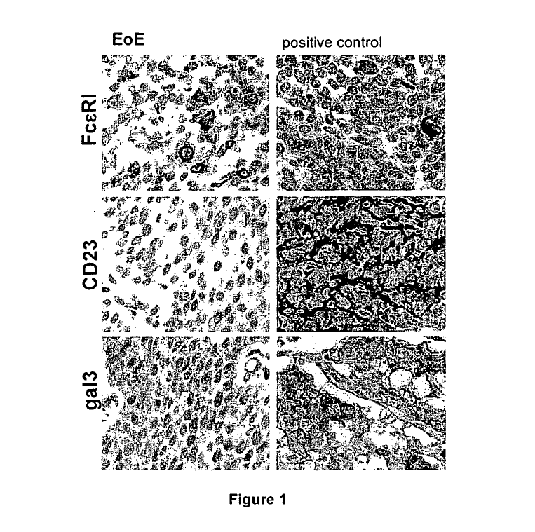

Unlike in the mouse, human FcεRI is also constitutively expressed on the surface of eosinophils, dendritic cells, macrophages and neutrophils. Up until recently, the type of IgE receptors and the cells that express those receptors in the esophagus of EoE subjects were poorly defined. Based on the expression pattern of FcεRI on peripheral blood cells and expression profiling data from EoE subjects, it was hypothesized then was later confirmed that FcεRI is highly expressed in tissue lesions of subjects with EoE. It was demonstrated by the inventors of the present disclosure that FcεRI is actually the primary IgE-binding structure on inflammatory cells in tissue lesions from subjects with EoE (FIG. 1). The primary cell type that expresses FcεRI was defined as Langerhans cells, a highly immune modulatory type of antigen presenting cells. Receptor expression was significantly upregulated in EoE tissue when compared to tissue from healthy controls.

Accordingly, described herein is a means to carry out broad screening to define new effector molecules of Th2-mediated immunity. Additionally, the present disclosure is useful for investigating the general problem of Th2-mediated immune responses in humans. The basic mechanisms of how the immune system elicits Th2 immune responses are far less understood than the more dominant Th1 pathway. As discussed herein, it was discovered that FcεRI-mediated antigen presentation skews the immune response towards Th2 responses. Therefore, screening assays described herein may present a suitable tool to examine factors involved in the regulation of Th2 immune responses.

Previously, no cell models for studying the function of trimeric human FcεRIαγ₂ receptor existed. In fact, the only cell line to study the FcεRI receptor was RBL-2H3, which was used for virtually all studies of tetrameric FcεRIαβγ₂. The studies that originally described human trimeric FcεRIαγ₂ as an antigen uptake receptor were limited in their ability to detail the underlying immunological mechanisms due to the low numbers of primary cells available for detailed biochemical and morphological analysis. We established several new cell model systems, which allow various comparative studies of human tetrameric FcεRIαβγ₂ and trimeric FcεRIαγ₂. The Examples provide more detailed descriptions of these cells.

Based on the surprising discovery that a soluble form of FcεRI (sFcεRIα) can be used as a biomarker for IgE-mediated disorders in a subject, the disclosure also contemplates assays for detecting or measuring a soluble form of sFcεRIα in a sample. These assays are useful for identifying effector molecules that are involved in receptor activation and signaling thereof. These assays provide a tool for screening molecules that interact with FcεRI, IgE or both. These molecules are likely candidates for biomarkers of IgE-mediated disorders.

FcεRI is an activating immune receptor of the immunoglobulin superfamily, like other Fc receptors such as CD32, CD16, CD64 or CD89. The alpha chains of all these receptors carry the ligand-binding domain of the protein complex. These alpha chains use associated signaling subunits to induce immune activation after Ig-mediated receptor activations. For all of the above Fc receptors, excluding FcεRI, soluble forms of the alpha chains are described in the literature. Current understanding is that these sFc receptors can modulate Ig-mediated activation of the immune system. It is well described in the art that the immune system uses a soluble form of CD23 to modulate IgE-mediated immune responses. The idea that a soluble form of FcεRI existed was thus compelling, but previously a proof for the existence of this protein was lacking.

We found that FcεRI can exist as a soluble form in vivo in some subjects. Thus, in some embodiments, the presence of a soluble form of FcεRI indicates IgE-mediated disorders, regardless of serum IgE levels or clinical symptoms of allergic conditions. This is exemplified in an eosinophilic esophagitis model.

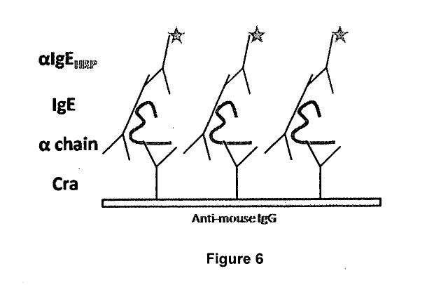

The presence of a soluble format FcεRI was first demonstrated in culture supernatants of the MelJusoαγ cells, which are described elsewhere herein. The cell line was established to study the function of trimeric FcεRI. An ELISA was established to allow for a quantification of this soluble protein. FIG. 6, shows a scheme of the sandwich ELISA used for detection.

The detection of sFcεRI in the serum of subjects faces the problem that a certain amount of this soluble protein might exist as a complex with serum IgE. This is particularly important with regards to the allergic status of the majority of EoE subjects because these subjects have high IgE levels. To be able to detect soluble FcεRI-IgE complexes as well as the free form of soluble FcεRI, the following assay was established: As a capturing antibody, we use mAb CRA-1. This mAb reacts with an epitope in the stalk region of the alpha-chain and does not interfere with IgE-binding. Soluble receptor that is bound to its specific Ab is then detected with human IgE and an anti-human IgE reagent. By omitting the IgE incubation step and performing detection with anti-human IgE reagent directly, our method allows also for quantification of the soluble FcεRI-IgE complexes which are present in the serum. A comparison of the signal with and without the additional IgE incubation step allows us to determine how much of the soluble FcεRI is complexed and how much is actually free in the serum.

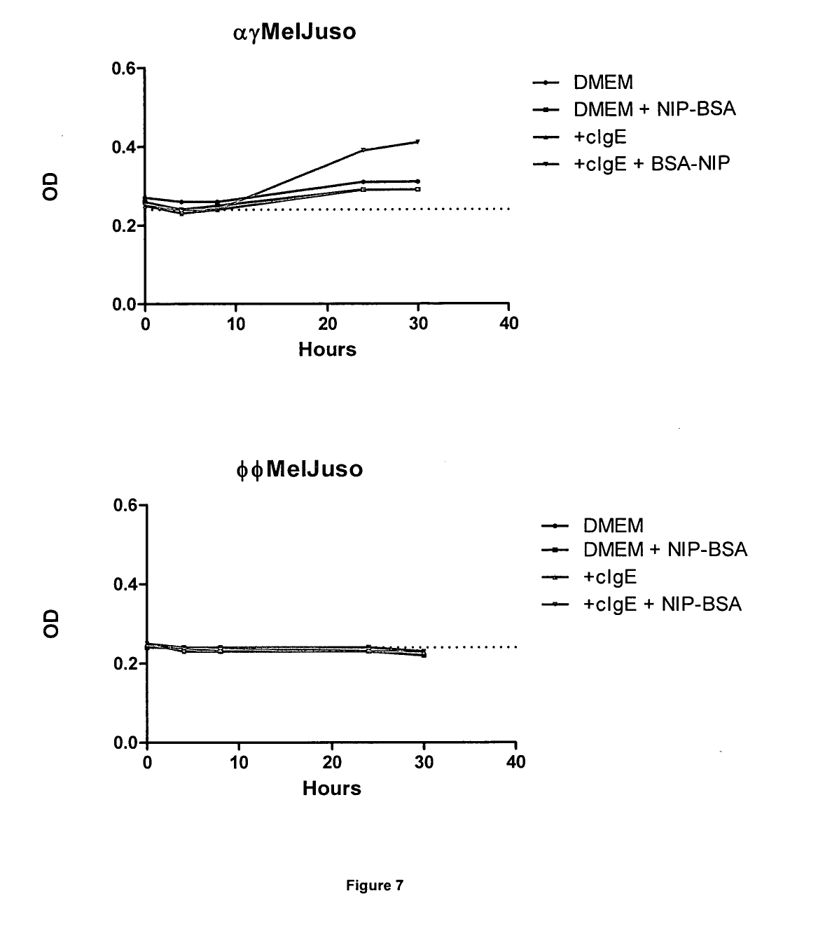

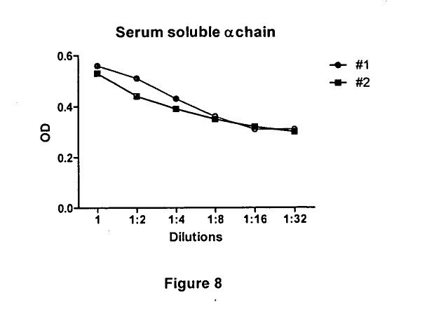

In the first set of experiments soluble FcεRI is detected in the cell culture supernatant of MelJusoαγ cells 24 h after the receptor was activated via IgE and antigen. MelJusoαγ were loaded over night with chimeric IgE that has the human Fc part and recognizes the hapten nitrophenol (NP) with its Fab fragment (cIgE). After removal of the excess cIgE in the culture supernatant, surface FcεRI was activated with haptenized antigen (NP-BSA). Aliquots of the supernatant were harvested at time point 4, 8, 24 and 32 h. FIG. 7 shows a quantification of soluble FcεRI in the supernatant of MelJusoαγ cultures with the new ELISA. Our analysis showed steady increase of protein over time. In a next set of experiments we analyzed whether we would be able to detect any soluble FcεRI in subjects with EoE. FIG. 8 shows a dilution curve with serum of a representative subject. We conclude from this experiment that we can detect soluble FcεRI in subjects' serum and that we are also able to perform quantitative analysis of serum levels of soluble FcεRI.

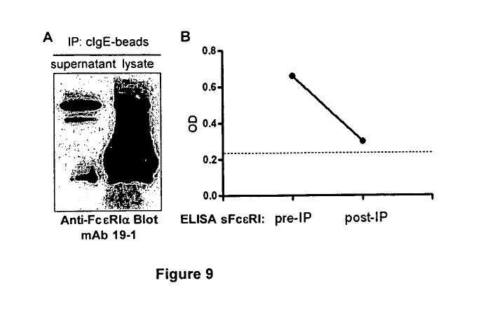

When soluble FcεRI is precipitated from cell culture supernatants with cIgE and is subjected to Western blot analysis with the alpha-chain specific mAb 19-1 (shown in FIG. 9), the molecular weight of the resulting band of immunoreactivity is similar to that of sFcεRI alpha-chain expressed by the cells.

To further confirm that sFcεRI is released into supernatant, a comparative analysis may be performed to examine serum levels of sFcεRI in a retrospective study with sera collected from subjects with IgE-mediated disorders, such as EoE, reflux esophagitis and healthy controls.

The collection of sera from subjects may be further examined to perform a comparative analysis of levels of IgE, sFcεRI and IgE-sFcεRI complexes. Thus, the instant disclosure also includes assay systems that are useful to quantify the levels of IgE, sFcεRI and IgE-sFcεRI complexes. Embodiments drawn to these assays are described in more detail below.

In some embodiments, quantitative PCR may be combined to assess the gene expression of the FcεRI receptor in a sample and to normalize sFcεRI protein levels in the sample against the expression level. In some cases, sFcεRI levels are monitored during diagnosis and/or therapy of an IgE-mediated disorder. In some embodiments, a prospective study may be carried out to compare the sFcεRI levels to FcεRI receptor expression levels in affected lesions and/or on peripheral blood samples. For example, esophageal biopsies may be collected from EoE subjects, and the specimen is used to perform quantitative PCR to assess the alpha chain and the beta chain of FcεRI.

The secretion (or release) of sFcεRI is dependent on activation of the receptor. Thus, it is of interest to examine the mechanism that underlies the production of sFcεRI. Because the soluble form of the protein may modulate IgE-responses in vivo, it is important to understand the signaling events and the mechanism that is involved in its production and secretion.

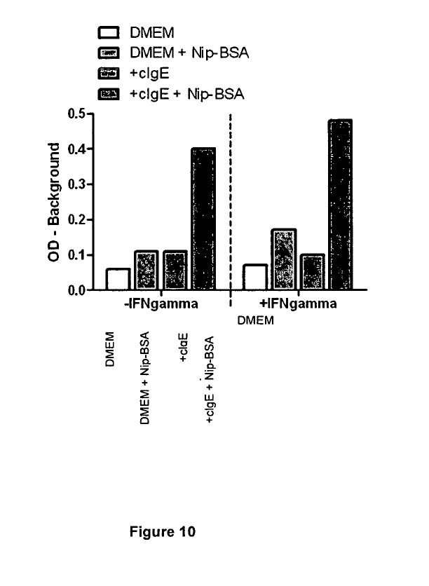

Accordingly, the present disclosure also includes a method of identifying a candidate molecule that regulates FcεRI expression. In one embodiment, the candidate molecule is a cytokine. To illustrate, experiments were carried out to examine whether the production of sFcεRI may be modulated. IFN-γ was selected as a likely candidate to modulate the production of sFcεRI because this cytokine is a well-described down-modulator of FcεRI expression at the cell surface. When we compared the production of sFcεRI in the absence and presence of IFN-γ it was observed that the presence of this cytokine enhances production of sFcεRI (see FIG. 10). IFN-γ was shown to modulate the production of sFcεRI, supporting the idea that the production of sFcεRI can be modified by an immune modulatory cytokine. One possible mechanism of how to decrease FcεRI receptor expression might be to induce shedding of the protein from the cell surface. Mechanisms that decrease surface expression of FcεRI are important to understand because the IgE-mediated immune response correlates to IgE-binding sites at the cell surface.

In the next set of experiments, a number of defined stimuli of the immune system are evaluated for their ability to modify secretion (e.g., release) of sFcεRI. A panel of cytokines and chemokines relevant for allergy and Th2 mediated immune responses are included for analyses, such as TSLP, IL4, IL5 and IL6. Toll receptor ligands, such as LPS and flagellin. Elucidation of the signal transduction pathways involved in the production of sFcεRI is also of interest. Therefore, the production of sFcεRI may be tested in the presence of chemical inhibitors defined for particular pathways. Broad kinase inhibitors, inhibitors of metalloproteases and cathepsin inhibitors are added to the MelJusoαγ cells during the production of sFcεRI. With this set of experiments, initial information about the signaling mechanisms involved in production of the soluble protein are obtained.

Based on the teaching of this disclosure, one may now perform a broad screen for markers that depend on IgE-FcεRI activation of dendritic cells with a special focus on molecules that can potentially regulate tissue remodeling, such as, for example, in the esophagus.

Evidence shows that FcεRI-mediated antigen presentation skews immune responses towards Th2. This finding and our observation that FcεRI is the major IgE-binding structure in EoE lesions indicate the importance for a broad analysis of Th2-type cytokines patterns and for quantification of their expression levels during the course of EoE. Data further suggest that tissue-remodeling events in EoE are also connected to the allergic phenotype of the disease. New molecules that mediate these Th2 responses may represent candidates for marker proteins of EoE and other IgE-mediated disorders. With this broad targeted screen, new predictive markers for chronicity and long-term complications of EoE and other IgE-mediated disorders may be effectively identified.

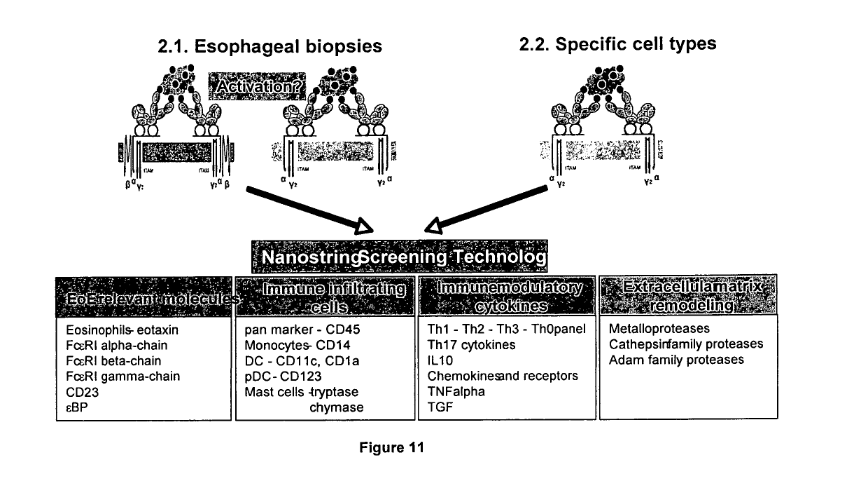

The Nanostring Technology may be used for carrying out the screening. Briefly, this nCounter Analysis System™ uses a novel digital technology that is based on direct multiplexed measurement of gene expression and offers high levels of sensitivity (500 attomolar <1 copy per cell) and precision. The technology uses single molecule imaging and high numbers of unique transcripts in a single reaction. Such technology is critical given the small amount of RNA available for analysis from tissue biopsies, such as esophageal biopsies. The high sensitivity of the method is also important for analysis to evaluate new marker proteins for their potential to be detected in selective tissues or cell populations, such as human dendritic cells, serum or on peripheral blood cells. This technology allows for the analysis with as few as 500 cells and it is significantly easier to generate a highly homogenous cell population in lower rather than higher cell numbers. Another advantage of this analysis is the ability for the user to custom design chips based on preferred genes of interest. Up to 500 different genes may be studied and obtain information such as copy number per cell. FIG. 11 shows the principal strategy for our screen. Targets of interest are divided into groups and marker proteins for each of these groups are defined. This scheme shows a minimal panel of investigation and allows for further modification once certain proteins are excluded as markers based on a comparison with control condition, in this case, such as reflux esophagitis.

An mRNA Screen for the Consequences of IgE-FcεR-Mediated Activation on Cytokine and Growth Factor Induction on Defined Cell Populations In Vitro

It is feasible to combine the screening assays described in the present disclosure with a comparative quantitative mRNA analysis of cytokines, chemokines and tissue remodeling factors in esophageal biopsies from subjects with EoE or reflux esophagitis, as well as healthy controls.

The analysis described here should provide a picture of the situation in the esophageal tissue with regards to markers of immune inflammatory events in EoE subjects. Not necessarily all of the molecules resulting from the tissue screen have to depend on IgE-FcεR-mediated activation of the immune system. Rather, it is likely that additional mediators that are more generally associated with the EoE pathology are also identified by this method. Additional screen with defined cells and defined conditions of FcεR-mediated cell activation may help to distinguish between receptor-dependent and independent marker proteins in the subjects.

A comparative screen is useful to discern IgE-FcεR-mediated activation events in human mast cells, Langerhans cells and eosinophils. These cells are cultured according to established methods and FcεRI is loaded and activated as described herein in this application. It is also feasible to isolate and culture T cells from EoE tissue lesions for further investigation. All methods are already established in the art. Additional analyses include the effect of TSLP, a classical mediator see during allergic responses, and Toll receptor ligands on the detected mRNA patterns.

### Data Analysis

Chi-square is used to compare the proportion of positive markers between EoE and controls. A comparison between the proportion of positive markers between EoE and those with reflux disease, controlling for the level of inflammation, is also performed. Biomarker levels in body fluids will be correlated with disease activity as described and with biomarker levels obtained from extracts of biopsy samples. Comparison among groups is achieved by repeated measures analysis.

In summary, FcεRI is a critical structure in triggering eosinophilic inflammation in IgE-mediated disorders, such as EoE. Therefore, FcεRI and effector molecules of FcεRI-mediated immune events are potential targets for the discovery of new biomarkers. This notion is supported in several ways. Numerous case reports and clinical studies have pointed out the strong association between EoE and atopic diseases. Gene expression profiling of EoE subjects showed upregulation of FcεRI. The inflammatory infiltrate in EoE is characterized by eosinophils, mast cells, and dendritic cells of the Langerhans cell type. All of these cells abundantly express FcεRI. It is therefore highly likely that FcεRI is critically involved in the pathology of EoE. Based on these observations, and based on the data presented herein, a new soluble form of this IgE-receptor, sFcεRI, has emerged as a biomarker and a broad targeted screen for molecules downstream of FcεRI-mediated cell activation.

Embraced in the instant disclosure are assay systems to detect IgE receptors. In particular, the disclosure includes, but is not limited to, ELISA for quantification of the alpha chain of the human high affinity IgE receptor. The assays provided in the disclosure offer clinical applications for IgE-mediated disorders. More specifically, the assays described herein are used to detect and/or monitor IgE-mediated disorders, e.g., allergic conditions. For example, such ELISA-based quantification provides a useful means for following protein expression in cell lysates derived from peripheral blood as well as other body fluids. Additionally, this test provides a means for investigating whether this protein is a marker for other diseases.

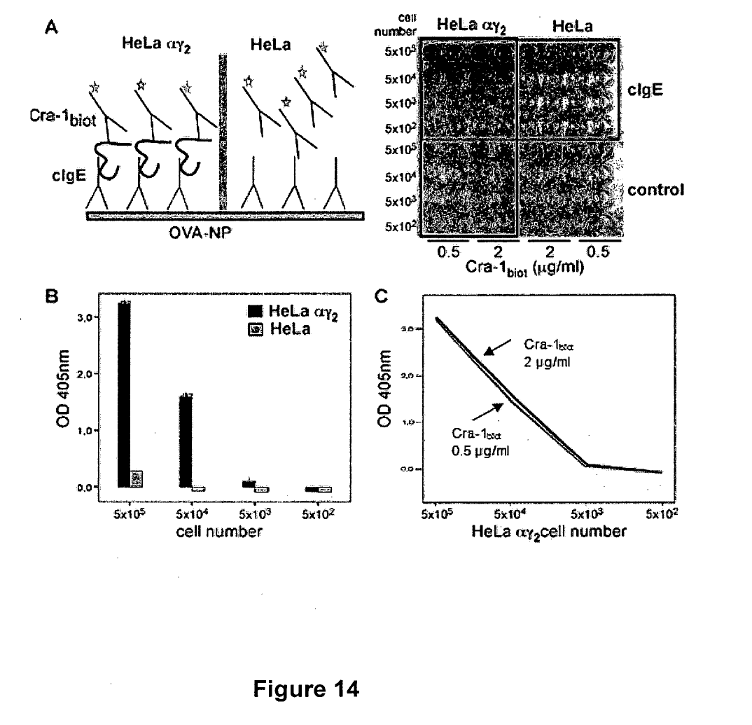

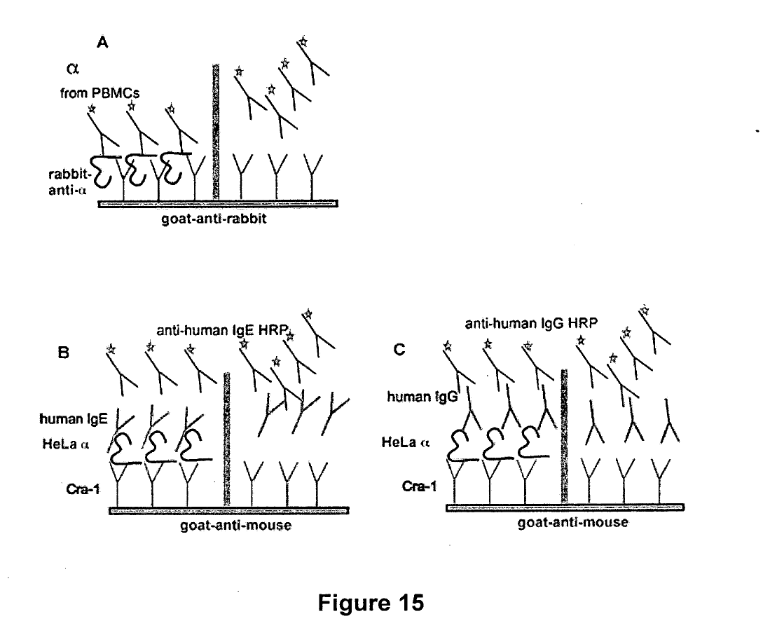

In one embodiment, the assay is an ELISA for the quantification of an IgE-binding protein. In some cases, the assay relates to detecting atopy. An exemplary embodiment of such an assay is illustrated in FIG. 14. In some embodiments, the assays are directed to detecting confirmation-specific FcεRI. For example, the assay allows specific (i.e., selective) or preferential detection of properly folded alpha protein. In another embodiment, the assay allows the detection of the alpha protein irrespective of its folding stage, e.g., non-conformational specific. Non-limiting examples of modifications of the protocol are illustrated in FIG. 15B. In yet further embodiment, modifications (FIG. 15C) of the test allow for use as an anti-human IgE detection ELISA and an ELISA for IgG auto-antibodies against this protein (FIG. 15D).

As illustrated in FIGS. 14 and 15, the assays of the instant disclosure typically comprise a solid substrate onto which appropriate binding factor(s) are coupled. The coupling between specific immune complexes may be direct coupling or indirect coupling. The term “solid substrate” is not intended to be limiting; however, in some embodiments, the solid substrate is a plastic plate suitable for ELISA analyses. A working example of such application is provided in Examples below; See, for example, FIG. 16 for illustration.

### EXAMPLES

### Materials and Methods

### Antibodies and Reagents

Anti-human FcεRI alpha mAb 19-1 was kindly provided by Dr. J.-P. Kinet (Laboratory of Allergy and Immunology, Beth Israel Deaconess Medical Center, Boston, Mass.) and used as previously described^{24, 37-38}. Anti-human FcεRI alpha mAb CRA1 (clone AER-37) was purchased from eBioscience, San Diego, Calif. Anti-FcεRI-gamma polyclonal serum was purchased from Millipore, Billerica, Mass. Chimeric IgE that contains the human Fc domain and recognizes the haptens 4-hydroxy-3-nitrophenylacetic acid (NP) and 4-hydroxy-3-iodo-5-nitrophenylacetic acid (NIP) with its Fab region (cIgE) was derived from Jw 8/5/13 cells (kindly provided by Dr. D. Maurer, Department of Dermatology, Medical University of Vienna, Austria) and was used for immunoprecipitation of properly folded FcεRI-alpha and for in vitro cell culture experiments. Phycoerythrin (PE)-conjugated was purchased from Biosearch Technologies, Novato, Calif., and used for flow cytometry analysis. Allophycocyanin (APC) conjugated anti-human FcεRIα mAb CRA1 and the appropriate mIgG2b isotype control mAbs were purchased from eBioscience and used for FACS analysis. Anti-mouse IgG (Fc specific, produced in goat; Sigma Aldrich, St. Louis, Mo., #M3534-1 mL) was used for coating of the ELISA plates. High-IgE human serum was purchased from Bioreclamation, Hicksville, N.Y. and used for detecting captured sFcεRI by ELISA. Goat anti-human IgE HRP conjugated antibody (Caltag, Invitrogen, Carlsbad, Calif.) was used as a secondary antibody.

### Cell Lines and Culture Media

MelJuso cells that stably express FcεRI-alpha and FcεRI-gamma (MelJusoαγ) were generated by viral transduction according to standard protocols (http://www.stanford.edu/group/nolan/). For transfection of MelJuSoαγ cells with a FcεRI-alpha cDNA construct, <NP_{—}001992.1> was used. MelJuSo empty vector and MelJuSoαγ cells were maintained in Dulbecco's minimal essential medium (DMEM, Cellgro, MediaTech, Herndon, Va.) supplemented with 10% fetal calf serum (HyClone, Logan, Utah), 2 mM glutamine (Cellgro), 100 U/ml Penicillin, and 100 μg/ml Streptomycin (Gibco BRL, Gaithersburg, Md.). Cells were reselected using hygromycin (1 mg/ml) and puromycin (0.5 μg/ml) Jw 8/5/13 cells were cultured in suspension and maintained in RPMI 1640 medium (Gibco, Invitrogen, Grand Island, N.Y.) supplemented with 10% fetal calf serum (HyClone, Logan, Utah), 2 mM glutamine (Cellgro), 100 U/ml Penicillin, and 100 μg/ml Streptomycin (Gibco BRL, Gaithersburg, Md.).

Collection of Sera from Subjects

Sera from ten adult volunteers were screened for the presence of sFcεRI by immunoblot and ELISA. Sera from eight poly-sensitized, highly atopic patients collected after IRB approval at the University of Vienna MUW were analyzed for sFcεRI. Total serum IgE and allergen-specific IgE were measured by a solid phase immunoassay (CAP-System, Pharmacia Diagnostics, Uppsala, Sweden). Total serum IgE levels are given in kU/l, specific IgE is given in CAP RAST classes.

Sera from 122 children were obtained from an ongoing prospective cohort study on the role of FcεRI in the gastrointestinal tract. Patients between 1 and 18 years of age scheduled for an elective esophago-gastro-duodenoscopy at the Division of Gastroenterology at Children's Hospital Boston were randomly invited to participate. Subjects who used steroids in any form, immunomodulatory drugs, mast cell stabilizer, or leukotriene inhibitor within the last 3 months, as well as patients with an established diagnosis of autoimmune, inflammatory, or immunodeficiency disease were not enrolled. The study protocol was approved by the Investigational Review Board at Children's Hospital Boston (Harvard Medical School, Boston, Mass.). Patients or their legal guardians provided written informed consent. Total serum IgE was measured using a solid-phase ELISA (DiaMed Eurogen, Turnhout, Belgium) according to the manufacturer's instructions as described elsewhere²⁵. Normal serum IgE levels are given by the manufacturer as <10 IU/ml for age 0-3 years, <25 IU/ml for 3-4 years, <50 IU/ml for 4-7 years, <100 IU/ml for 7-14 years, and <150 IU/mL for adults older than age 14.

### Receptor Activation and Production of Soluble Alpha in Cell Culture Supernatants.

MelJusoαγ cells were grown to confluence and incubated with cIgE over night. Excess cIgE was washed away and ligand-bound receptor was activated with haptenized antigen (BSA- or OVA-, 1 μg/ml, both Biosearch Technologies, Novato, Calif.). Cell culture supernatants were collected after the indicated time periods and analyzed for the presence of soluble alpha chain by ELISA or by immunoprecipitation.

### Removal of Exosomes

To remove exosomes from cell culture supernatants, MelJusoαγ supernatants were treated with a sequence of ultracentrifugation steps following the protocol published by Thery et al.^{27, 39}

### Flow Cytometry

MelJusoαγ were loaded with either a mix of cIgE:PBS buffer or cIgE:sFcεRI from serum or culture supernatants for 30 minutes on ice. FcεRI-bound cIgE was stained with phycoerythrin (PE)-conjugated (Biosearch Technologies) and analyzed on a BD FACScan™ flow cytometer using CellQuest software for acquisition and analysis (both from Becton Dickinson).

### Statistical Analysis.

Correlations between serum IgE and serum-sFcεRI were calculated by Spearman's rank correlation test using SPSS for Windows (version 16.0, SPSS Inc., Chicago, Ill.). Spearman's rank correlation coefficients are displayed as ‘rho’.

### Immunoprecipitation and Immunoblotting

Cells were solubilized in lysis buffer (0.5% Brij 96, 20 mM Tris, pH 8.2, 20 mM NaCl, 2 mM EDTA, 0.1% NaN₃) containing protease inhibitors (Complete, Roche) for 30 min on ice. Immunoprecipitation was performed with cIgE anti-NP (Serotec) and anti-NIP beads (Sigma) as previously described^{24, 40}. Proteins were eluted from beads in non-reducing Laemmli sample buffer and samples were run on 12% non-reducing SDS-PAGE gels, transferred to PVDF membrane (Pierce) and probed with anti-FcεRIα (mAb 19-1) followed by peroxidase-conjugated goat-anti-mouse IgG for detection of precipitated α-chain. In the case of the sFcεRI supernatants from MelJuSoαγ cells were collected and purified over an IgE column. Peroxidase activity was detected using SuperSignal.

### Developing Models for Studying IgE-FcεRI-Mediated Activation of Antigen Presenting Cells

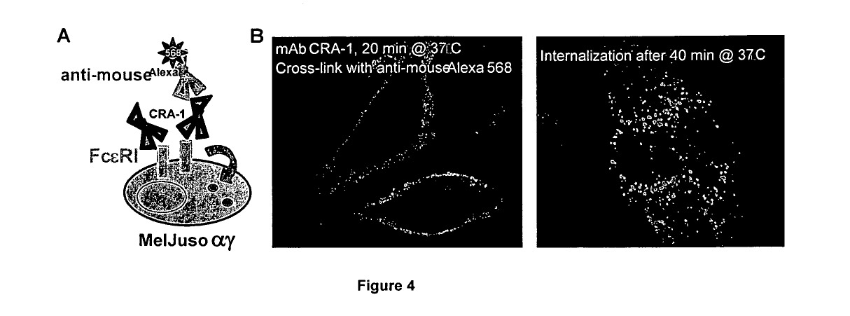

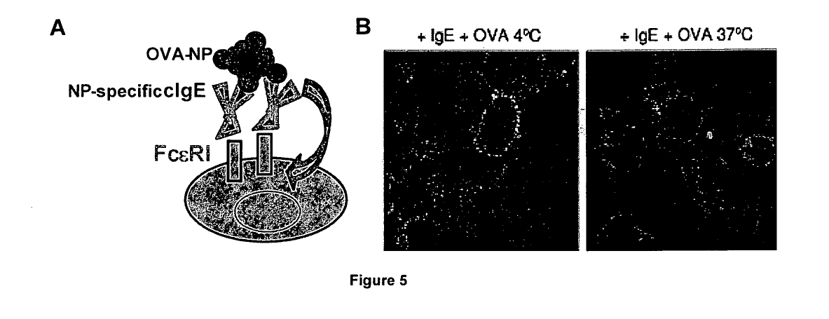

A MelJuSo cell line was established that expresses trimeric FcεRIαγ₂ (FIG. 4, cell line is referred to as αγMelJuSo or MelJuSoαγ). MelJuSo cells are well characterized and this line is commonly used as a model for human non-professional antigen presenting cells. FIG. 4 shows an FcεRIαγ₂ internalization experiment conducted with αγMelJuSo. As the interaction of IgE with FcεRIα is pH sensitive, we decided to activate the cells via receptor specific mAb rather than IgE and antigen (scheme FIG. 4A). We might have otherwise lost detection of the activated receptor in acidic compartments after internalization. Loading of the receptor with mAb CRA-1 for 20 min is followed by a second step crosslinking anti-mouse reagent, which is coupled to Alexa 568. Approximately 5 min after addition of the anti-mouse Alexa 568, surface engagement of the receptor becomes visible. This time point is defined as ‘zero’ and receptor internalization is followed by live cell microscopy (representative pictures, FIG. 4B). FIG. 5 shows a scheme of the IgE loading conditions and IgE-antigen-mediated receptor activation, which was established with αDC2.4, another FcεRIαγ₂ expressing cell line that we established. We modified this murine dendritic cell line to express trimeric FcεR at the cell surface, because this line is well-established line for professional antigen presenting cells. This method of loading allows for receptor specific internalization of antigen in the absence of pinocytotic antigen uptake. This method of loading and receptor activation method has worked with all cell types analyzed so far. IgE-mediated cell activation was used for our experiments that demonstrate the production of sFcεRI by αγMelJuSo cells.

### Results

A Soluble Form of FcεRI Alpha is Produced after IgE-Antigen-Mediated Receptor Crosslinking

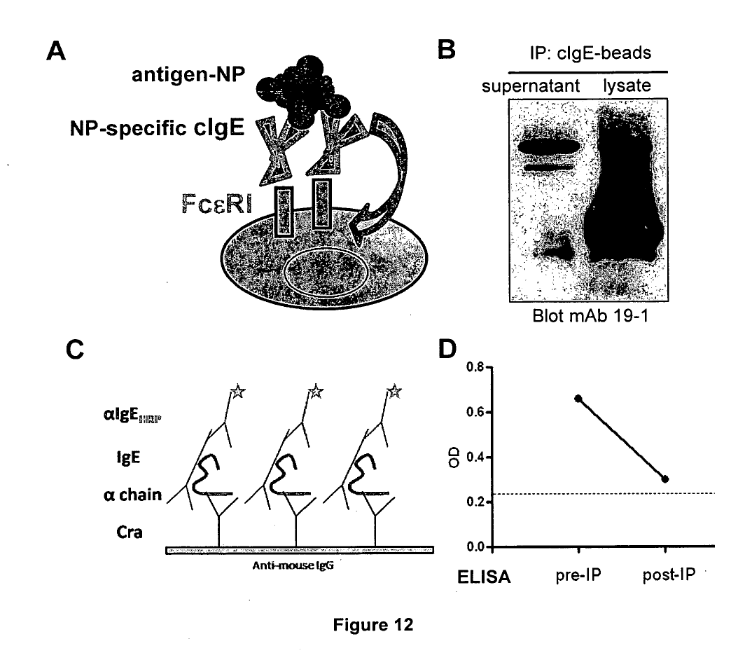

Several new cell lines were established to study the function of trimeric FcεRI in vitro. These new research tools were used to address the question as to whether IgE-mediated activation of surface FcεRI induces the release of a soluble form of the receptor into culture supernatant. MelJuSoαγ were loaded over night with chimeric IgE that contains the human Fc part and recognizes the hapten nitrophenol (NP) with its Fab part (cIgE). After removal of excess cIgE, surface FcεRI was activated by crosslinking the receptor-bound ligand with haptenized antigen (NP-BSA or NP-OVA). A scheme for receptor activation is provided in FIG. 12A. 36 h after receptor activation, a cIgE column was used to retrieve soluble alpha chain protein from culture supernatants. Precipitates were analyzed with the alpha-chain specific mAb 19-1 by western blot analysis. FcεRI alpha in supernatants shows a different molecular weight characteristic as compared to FcεRI alpha in cells (FIG. 12B). The precipitated protein is comparable in molecular weight to the sFcεRI pulled down from patient sera (FIG. 12B). The apparent molecular weigh of about 30-40 kDa (broad range of molecular weight is explained by the highly glycosylated nature of the protein) indicates that the sFcεRI present in the supernatant is a cleaved shorter version of the protein (FIG. 12B).

To be able to quantify sFcεRI, the following ELISA assay was established: mAb CRA-1 was used as a capturing antibody on plates coated with polyclonal anti-mouse IgG. CRA-1 reacts with an epitope in the stalk region of FcεRI alpha and does not interfere with IgE-binding. sFcεRI that is captured by its specific mAb is then detected with cIgE and an anti-human IgE reagent (FIG. 12C). We used this ELISA to analyze the presence of sFcεRI supernatants of activated MelJuSoαγ prior and post immunoprecipitation. IgE immunoprecipitation depleted the culture supernatant from sFcεRI protein (FIG. 12D).

Kinetics of sFcεRI Accumulation in Culture Supernatants

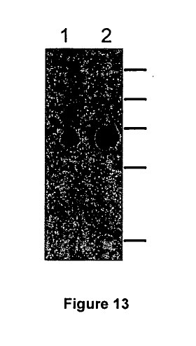

In the first set of experiments we were able to detect sFcεRI in the cell culture supernatant of MelJuSoαγ cells 36 h after the receptor was activated. Next we studied the accumulation of the soluble form in supernatants in a time course. Aliquots were harvested 4, 8, 24 and 32 h after receptor activation. FIG. 7 shows a quantification of sFcεRI in the supernatant of MelJuSoαγ cultures by ELISA. Our analysis showed steady increase of protein over time approaching a plateau after about 24 h (FIG. 7A). As another specificity control we showed that sFcεRI could not be detected in supernatants of the empty vector-transfected maternal cell line that does not express FcεRI (FIG. 7B). To prove that the detected protein was a genuine soluble form of FcεRI and not transmembrane FcεRI, sequential high-speed ultracentrifugation was performed to deplete the supernatants from cell debris and exosomes as established by Thery et al.²⁷ sFcεRI was detected in exosome-depleted culture supernatants confirming that the detected protein is as a bona fide soluble version of the receptor. (See FIG. 13).

Production of sFcεRI is Modulated by INFγ

We so far described the secretion of sFcεRI is dependent on activation of the receptor. In the next set of experiments, we studied whether the production of sFcεRI can be modulated. We studied the influences of IFN-γ on the production of sFcεRI. IFN-γ appeared as a promising candidate because this cytokine is a well-described down-modulator of FcεRI expression at the cell surface. When we compared the production of sFcεRI in the absence and presence of the cytokine, we saw that IFN-γ enhances production of sFcεRI (FIG. 10). The ability of IFN-γ to modulate the production of sFcεRI proves that the production of sFcεRI can be modified by an immune modulatory cytokine.

Detection of a Soluble Form of FcεRI Alpha (sFcεRI) in Human Serum by Immunoprecipitation

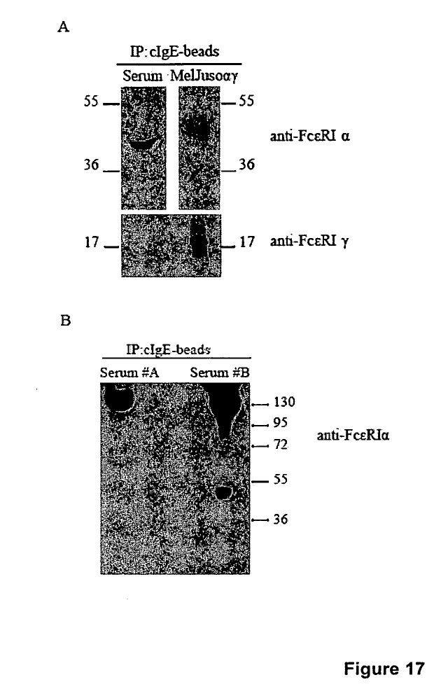

To investigate whether a soluble form of the alpha chain of FcεRI exists in humans, we used the receptor's natural ligand IgE as a bait to perform immunoprecipitations from serum of randomly selected adult volunteers (representative individuals, n=10, FIG. 17 and data not shown). Precipitates from cIgE-columns were analyzed with the alpha-chain specific mAb 19-1 by Western blot and compared to FcεRI alpha precipitated from the cell surface of MelJuSoαγ cells (FIG. 17A). A soluble form of FcεRI (sFcεRI) was precipitated from human serum as a protein of ˜30-40 kD. As expected for a soluble form, sFcεRI has a lower molecular weight than the surface expressed protein. Unlike transmembrane FcεRI-alpha that forms a multimeric complex with the common FcR-gamma chain 1, 5, sFcεRI does not associate with FcR-gamma (FIG. 17A). Another finding was that not every individual tested had circulating sFcεRI (FIG. 17B, left lane).

Detection of sFcεRI in Human Serum by ELISA

To quantify sFcεRI in human serum, we established a sandwich-ELISA system with anti-FcεRI mAb CRA1 as capture antibody. CRA1 binds the stalk region of the alpha-chain and does not interfere with the IgE-binding site of FcεRI. We thus used human IgE to bind to mAb-captured sFcεRIIα and followed with a goat anti-human IgE-HRP conjugate for detection (FIG. 6). Using this approach we detected sFcεRI in sera of eight atopic patients (5 boys, 3 girls, mean age 10.3+/−2.7 years, Table 1). FIG. 8 shows a dilution curve with serum of two allergic patients. Based on our dilution curve we set the cut of for detection around an 0.2 OD in our ELISA. We were also able to detect sFcεRI in plasma (data not shown) and further confirmed that sFcεRIIα itself did not interfere with detection of IgE (data not shown). In summary, this set of data describes the semi-quantitative analysis of serum levels of sFcεRI with the newly established ELISA system.

As illustrated in FIG. 14A, cIgE anti-NP was coupled to a NIP-OVA-precoated plate. After ON blocking, plates were reacted with serial dilutions of NP-40 cell lysates of HeLaαγ or untransfected HeLa cells at the indicated concentrations. Binding of sFcεRI to its natural ligand IgE was detected with biotinylated mAb Cra-1 and Streptavidin-HRP. FIG. 14B depicts comparison of optical density (OD) measured at 405 nm from serial dilutions of cell lysates. FIG. 14C shows that the sensitivity of the Cra-1 antibody was maintained at the two concentrations tested (2 μg/ml and 0.5 μg/ml) within the range of dilutions as indicated.

Serum Levels of sFcεRI Correlate with Serum IgE Levels in Patients with Elevated IgE

We next investigated the occurrence of sFcεRI in a heterogeneous pediatric population screening sera from a cohort of 122 children (mean age 10.68+/−5.26 years). Like most serum-parameters, serum levels of sFcεRI showed a right skew among our study population, with a median OD of 0.09 and a range from zero to 1.38 (FIG. 16B). We also found that in children with elevated serum IgE, sFcεRI serum levels correlated with serum IgE levels (rho=0.413, Spearman's rank correlation, FIG. 16C). No correlation was found in non-atopic subjects (n=97, Spearman's rho, right panel) or the total study population (n=122, Spearman's rho=0.065, data not shown). As shown in FIG. 16D, some subjects have high serum alpha levels despite normal serum IgE titers.

sFcεRI Circulates as a Free or an IgE-Complexed Protein in Human Serum

The sFcεRI has a high affinity binding site for IgE. It is thus likely that human serum the soluble form of the receptor as a complex with IgE. By omitting the IgE incubation step in our assay and performing detection with anti-human IgE reagent directly, our method allows also for the quantification of the sFcεRI-IgE complexes which were preformed in serum already. A comparison of the signal with and without the additional IgE incubation step allows us to determine how much of the sFcεRI is complexed and how much is circulating in its free form in serum. We found that in human serum sFcεRI is present as both a free and an IgE-complexed protein (FIG. 16E).

sFcεRI Inhibits IgE Loading of FcεRI at the Cell Surface

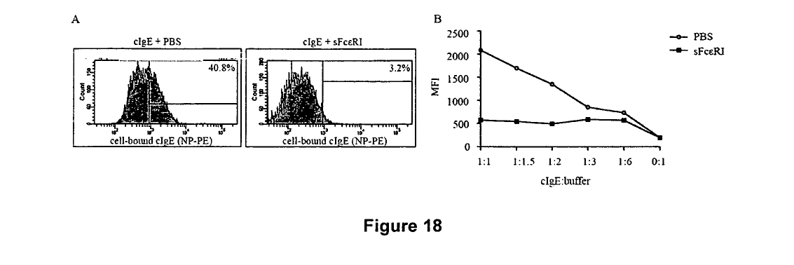

Since we detected IgE-sFcεRI complexes in serum, we speculated sFcεRI could interfere with IgE-binding to FcεRI when expressed at the cell surface. If so, sFcεRI could function as a potential modulator of IgE-mediated immune activation. We tested this hypothesis by loading FcεRI-expressing MelJuSoαγ with either a 1:2 mix of cIgE and donor serum containing high sFcεRI levels or with cIgE diluted with PBS as a control. Cell-bound cIgE was visualized by flow cytometry with PE-conjugated-hapten. sFcεRI in the serum efficiently blocked cIgE binding to cellular FcεRI (FIG. 18A). Similarly, sFcεRI from supernatants of MelJuSoαγ cells inhibited IgE-loading of FcεRI expressed at the surface of cells (FIG. 18B). These results demonstrate that sFcεRI has the potential to inhibit IgE-loading of FcεRI at the cell surface of immune cells.

ELISA for the Detection of sFcεRI

ELISA plates were coated with anti-mouse IgG (Sigma Sciences, #M3534) and anti-alpha chain mAb (Cra, clone AER-37; eBioscience #14-5899-82) and incubated with patients' sera. After repetitive washing, bound alpha chain was loaded with its natural ligand IgE (Bioreclamation #HMPLCIT-IgE) and detected with goat anti-human IgE-HRPO Conjugate (Caltag #1115707). Conversion of substrate (3,3′,5,5′-Tetramethyl-benzidine Liquid Substrate for ELISA; Sigma #T0440) was measured at 450 nm. In a subset of patients, sFcεRI was measured in plasma and serum in parallel. For conversion of plasma samples into serum, BD Serum Separation Tubes (Becton Dickinson) were used according to the manufacturer's guidelines. A typical protocol for soluble alpha chain ELISA and a list of reagents are provided below:

1. Coat Plates
Dilute 2.5 mg/mL anti-mouse IgG (Fc Specific) 1:1000 in Na₂CO₃/NaHCO₃ coating buffer (100 mM)
For one plate: 20.98 mL Coating Buffer+21 μL anti-mouse IgG (Fc Specific)
Add 200 mL to each well of ELISA plate
Incubate over night @ 4° C.

2. Wash 3× (PBS+0.05% tween)

3. Incubate with CRA
Dilute CRA 1:1000 in Coating Buffer
For one plate: 20.98 mL Coating Buffer+21 μL CRA
Add 200 μL per well
Incubate all day @ 4° C.

4. Wash 3× (PBS+0.05% tween)

5. Block Plates
Use 10% FBS/PBS
For one Plate: 28.8 mL PBS+3.2 mL FBS
Add 300 μL to each well
Incubate overnight @ 4° C.

6. Wash 3× (PBS+0.05% tween)

7. Patient Serum
Dilute Serum 1:10 in PBS
For each sample: 22 uL Serum into 198 uL PBS
Add 100 uL undiluted to duplicate wells
Add 100 uL diluted serum to duplicate wells
For control use αγHeLa lysates in duplicate
Incubate for 90 min @ RT

8. Incubate with high IgE Serum
Dilute serum 1:10 in 0.5% FBS/PBS
For one plate: (18.8 mL PBS+94.5 uL FBS)+2.1 mL serum
Add 200 μL to each well
Incubate for 30 min @ RT

9. Wash 3× (PBS+0.05% tween)

10. Goat Anti-Human IgE-HRP
Dilute 1:1000
For one plate: 16.5 μL Anti-Human IgE+16.48 mL 10% FBS/PBS
Add 150 μl per well
Incubate for 30 min @ RT

11. Wash 6× (PBS+0.05% tween)

12. Substrate
TMB for ELISA
11 mL per plate
Add 100 mL per well
Incubate for ˜15 minutes (until color is dark enough)

13. Stop Reaction
Stop with 2N H₂SO₄
Dilute stock (18M) 1:18 in DW
for one plate: 325 μL H₂SO₄+5.5 mL Distilled Water
Add 50 μL per well

14. Read plate @ 450 nm

Reagents:
Coating Buffer: 100 mM Na₂CO₃/NaHCO₃, ph 9.6
Anti-mouse IgG (Fc Specific), produced in Goat; Sigma #M3534-1 mL; Lot#117K4784
Purified anti-human FcεRIα; clone AER-37; eBioscience #14-5899-82; Lot#E015324
Human Serum (Converted) (Sodium Citrate) Elevated IgE; Bioreclamation #HMPLCIT-IgE; Lot #BRH215527
Goat Anti-Human IgE (epsilon) HRPO Conjugate; Caltag #H15707; Lot #311347B
3,3′,5,5′-Tetramethyl-benzidine (TMB) Liquid Substrate for ELISA; Sigma #T0440-1L; Batch#028K0718

To our knowledge this study is the first description of a soluble serum form of human FcεRI, the high affinity receptor for IgE (sFcεRI). We here describe a soluble version of the FcεRI alpha chain that circulates in human serum as both a free protein and bound to its natural ligand IgE. Serum IgE and total sFcεRI levels correlate positively in individuals with elevated IgE levels. We further show that sFcεRI is released upon IgE-antigen-mediated activation of cell surface FcεRI in in vitro cultures. Most interestingly, sFcεRI from both human serum and cell culture supernatants interferes with IgE-binding to cellular FcεRI.

Commonly, the reagents used to detect FcεRI-alpha in its membrane bound form are directed against the IgE-binding epitope of the protein. Thus, the identification of sFcεRI could easily have been missed if the detection reagents were not selected carefully. We here established an ELISA system that uses a monoclonal antibody directed against the stalk region of the protein²⁸ to capture sFcεRI and use human IgE combined with anti-IgE for detection²⁴. By omitting the IgE incubation step, this ELISA also allows for an assessment of the amount of sFcεRI that circulates already in a complex with serum IgE.

So far, a single report has described a soluble FcεRI complex in cultures of human eosinophils²⁹. Since the integrity of FcεRI complexes requires the presence of cell membranes^{22, 26, 30}, Seminario et al. most likely described a version of the receptor that was released in an exosomal fraction rather than a bona fide soluble protein.

Based on our current understanding of the mechanism of sFcεRI generation, it is fair to postulate that serum sFcεRI is a reflection of FcεRI activation. In an independent study, we were able to confirm the observation of Liang et al.³¹ showing that patients can carry substantial amounts of IgE on peripheral blood cells even in the absence of elevated serum IgE. In summary, these two studies show that cells in the peripheral blood bind IgE from the serum and thereby can clear the serum of IgE. These IgE-loaded cells could be the source of sFcεRI when activated. Our finding that the presence of sFcεRI correlates with serum IgE supports this hypothesis. On the other hand, IgE-mediated cell activation could also account for the detection of serum sFcεRI in the absence of high serum IgE levels. sFcεRI is also an excellent candidate for an efficient in vivo modulator of IgE-mediated responses. While sCD23 has to trimerize to develop considerable affinity for its ligand 1, sFcεRI can bind IgE with a one-to-one ligand-receptor ratio. Additionally, the affinity of the FcεRI-IgE interaction is exceptionally high and disruption of a once formed contact requires low pH which is physiologically found only in the stomach^{1, 5, 7, 32}. The finding that receptor crosslinking is required for the production of sFcεRI also hints at a potential negative feedback mechanism. Antigen-IgE-mediated receptor crosslinking could induce shedding of sFcεRI to remove IgE-binding sites from the cell surface and to terminate receptor-mediated signalling. In addition, we show here that sFcεRI has the ability to prevent IgE-binding to surface expressed receptors. Thus the presence of serum sFcεRI could inhibit IgE-loading of effector cells of allergy in vivo.

Omalizumab is a recombinant humanized monoclonal antibody directed against serum IgE and currently approved for the treatment of severe allergic asthma³³⁻³⁵. Omalizumab also downregulates cell surface levels of FcεRI³⁶. It is believed that sFcεRI could prevent IgE-mediated activation of the immune system in a manner comparable to omalizumab.

TABLE 1
sFcεRI in sera of eight highly atopic patients.

| | | | Total | | |
|---|---|---|---|---|---|
| Pat. | | Age | IgE | | sFcεRI |
| ID | Gender | (years) | (kU/l) | Specific IgE (RAST class) | (OD) |
| 160 | f | 12.17 | 1318 | Timothy grass (5), rye (4), Birch pollen (6), | 0.57 |
| | | | | mugwort (2), cat (3), dog (2), cod (6), tuna | |
| | | | | (2), salmon (3), peanut (3), Hazelnut (3), | |
| | | | | paranut (3), almond (2), orange (2), coconut | |
| | | | | (2), apple (4), banana (2), peach (3), latex | |
| | | | | (2) | |
| 63 | f | 8.42 | 136 | Ragweed (3) Peanut (4) | 0.44 |
| 70 | m | 11.75 | 298 | Cat (3), milk (2), peanut (3), soy bean (2), | 0.59 |
| | | | | chicken (2) | |
| 368 | m | 9.7 | 733 | Birch pollen (6), house dut mite (2), dog (2), | 0.46 |
| | | | | peanut (2), hazelnut (6) | |
| 278 | f | 8.5 | 471 | Cat (3), dog (2), peanut (6), soy bean (3), | 0.67 |
| | | | | hazelnut (1), paranut (2) | |
| 463 | m | 14.7 | 104 | Timothy grass (4), rye (3), birch pollen (3), | 0.3 |
| | | | | mugwort (2), house dust mite (2), cat (3), | |
| | | | | egg (1), peanut (2), hazelnut (5), apple (2) | |
| 235 | m | 6.08 | 2465 | Timothy grass (4), rye (4), birch pollen (6), | 3.06 |
| | | | | mugwort (3), Cladosporium herbarum (3), | |
| | | | | house dust mite (3), cat (1), dog (2), egg (3), | |
| | | | | milk (2), fish (3), mussels (3), tuna (3), | |
| | | | | salmon (3), shrimp (4), wheat (3), peanut | |
| | | | | (6), soy bean (5), hazelnut (6), paranut (4), | |
| | | | | almond (3), coconut (3), | |
| 201 | m | 11.25 | 997 | Birch pollen (6), house dust mit (2), cat (2), | 0.92 |
| | | | | dog (2), egg (2), milk (3), wheat (3), rye | |
| | | | | (3), peanut (3), soy bean (3), hazelnut (6), | |
| | | | | paranut (3), almond (3), coconut (2) | |

f, female;
m, male;

### REFERENCES

1. Gould H J, Sutton B J. IgE in allergy and asthma today. Nat Rev Immunol 2008; 8:205-17.
2. von Bubnoff D, Novak N, Kraft S, Bieber T. The central role of FcepsilonRI in allergy. Clin Exp Dermatol 2003; 28:184-7.
3. Liu F T, Hsu D K. The role of galectin-3 in promotion of the inflammatory response. Drug News Perspect 2007; 20:455-60.
4. Abramson J, Pecht I. Regulation of the mast cell response to the type 1 Fc epsilon receptor. Immunol Rev 2007; 217:231-54.
5. Kraft S, Kinet J P. New developments in FcepsilonRI regulation, function and inhibition. Nat Rev Immunol 2007; 7:365-78.
6. Dehlink E, Fiebiger E. The role of the high-affinity IgE receptor, FcepsilonRI, in eosinophilic gastrointestinal diseases. Immunol Allergy Clin North Am 2009; 29:159-70, xii.
7. Kinet J P. The high-affinity IgE receptor (Fc epsilon RI): from physiology to pathology. Annu Rev Immunol 1999; 17:931-72.
8. Delespesse G, Sarfati M, Hofstetter H, Frost H, Kilchherr E, Suter U. Human Fc epsilon R II and IgE-binding factors. Int Arch Allergy Appl Immunol 1989; 88:18-22.
9. Aubry J P, Pochon S, Graber P, Jansen K U, Bonnefoy J Y. CD21 is a ligand for CD23 and regulates IgE production. Nature 1992; 358:505-7.
10. Bonnefoy J Y, Gauchat J F, Life P, Graber P, Aubry J P, Lecoanet-Henchoz S. Regulation of IgE synthesis by CD23/CD21 interaction. Int Arch Allergy Immunol 1995; 107:40-2.
11. Grenier-Brossette N, Bourget I, Akoundi C, Bonnefoy J Y, Cousin J L. Spontaneous and ligand-induced endocytosis of CD23 (Fc epsilon receptor II) from the surface of B lymphocytes generates a 16-kDa intracellular fragment. Eur J Immunol 1992; 22:1573-7.
12. Michel O, Kips J, Duchateau J, Vertongen F, Robert L, Collet H, et al. Severity of asthma is related to endotoxin in house dust. Am J Respir Crit. Care Med 1996; 154:1641-6.
13. Jung C M, Prinz J C, Rieber E P, Ring J. A reduction in allergen-induced Fc epsilon R2/CD23 expression on peripheral B cells correlates with successful hyposensitization in grass pollinosis. J Allergy Clin Immunol 1995; 95:77-87.
14. Delespesse G, Sarfati M, Heusser C. IgE synthesis. Curr Opin Immunol 1989; 2:506-12.
15. Delespesse G, Sarfati M, Hofstetter H. Human IgE-binding factors. Immunol Today 1989; 10:159-64.
16. Ishizaka K. IgE-binding factors and regulation of the IgE antibody response. Annu Rev Immunol 1988; 6:513-34.
17. Meuleman N, Stamatopoulos B, Dejeneffe M, El Housni H, Lagneaux L, Bron D. Doubling time of soluble CD23: a powerful prognostic factor for newly diagnosed and untreated stage A chronic lymphocytic leukemia patients. Leukemia 2008; 22:1882-90.
18. Reinisch W, Willheim M, Hilgarth M, Gasche C, Mader R, Szepfalusi S, et al. Soluble CD23 reliably reflects disease activity in B-cell chronic lymphocytic leukemia. J Clin Oncol 1994; 12:2146-52.
19. Sarfati M, Bron D, Lagneaux L, Fonteyn C, Frost H, Delespesse G. Elevation of IgE-binding factors in serum of patients with B cell-derived chronic lymphocytic leukemia. Blood 1988; 71:94-8.
20. Liu F T. Regulatory roles of galectins in the immune response. Int Arch Allergy Immunol 2005; 136:385-400.
21. Liu F T, Rabinovich G A. Galectins as modulators of tumour progression. Nat Rev Cancer 2005; 5:29-41.
22. Call M E, Wucherpfennig K W. Common themes in the assembly and architecture of activating immune receptors. Nat Rev Immunol 2007; 7:841-50.
23. van der Boog P J, van Zandbergen G, de Fijter J W, Klar-Mohamad N, van Seggelen A, Brandtzaeg P, et al. Fc alpha RI/CD89 circulates in human serum covalently linked to IgA in a polymeric state. J Immunol 2002; 168:1252-8.
24. Maurer D, Fiebiger E, Reininger B, Wolff-Winiski B, Jouvin M-H, Kilgus O, et al. Expression of functional high affinity immunoglobulin E receptors (FcεRI) on monocytes of atopic individuals. J. Exp. Med. 1994; 179:745-50.
25. Dehlink E. Relationships between levels of serum IgE, cell-bound IgE, and IgE-receptors on peripheral blood cells in a pediatric population. PlosOne 2010.
26. Fiebiger E, Tortorella D, Jouvin M H, Kinet J P, Ploegh H L. Cotranslational endoplasmic reticulum assembly of Fc{varepsilon}RI controls the formation of functional IgE-binding receptors. J Exp Med 2005; 201:267-77.
27. Thery C, Regnault A, Garin J, Wolfers J, Zitvogel L, Ricciardi-Castagnoli P, et al. Molecular characterization of dendritic cell-derived exosomes. Selective accumulation of the heat shock protein hsc73. J Cell Biol 1999; 147:599-610.
28. Kubota T, Mukai K, Minegishi Y, Karasuyama H. Different stabilities of the structurally related receptors for IgE and IgG on the cell surface are determined by length of the stalk region in their alpha-chains. J Immunol 2006; 176:7008-14.
29. Seminario M C, Saini S S, MacGlashan D W, Jr., Bochner B S. Intracellular expression and release of Fc epsilon RI alpha by human eosinophils. J Immunol 1999; 162:6893-900.
30. Singleton T E, Platzer B, Dehlink E, Fiebiger E. The first transmembrane region of the beta-chain stabilizes the tetrameric Fc epsilon RI complex. Mol Immunol 2009; 46:2333-9.
31. Liang Y, Ganley-Leal L M. A simple method for measuring human cell-bound IgE levels in whole blood. J Immunol Methods 2009.
32. Kinet J P. The essential role of mast cells in orchestrating inflammation. Immunol Rev 2007; 217:5-7.
33. Firszt R, Kraft M. Pharmacotherapy of severe asthma. Curr Opin Pharmacol 2010; 10:266-71.
34. Molimard M, Buhl R, Niven R, Le Gros V, Thielen A, Thirlwell J, et al. Omalizumab reduces oral corticosteroid use in patients with severe allergic asthma: real-life data. Respir Med 2010; 104:1381-5.
35. Rodrigo G J, Neffen H, Castro-Rodriguez J A. Efficacy and Safety of Subcutaneous Omalizumab Versus Placebo as Add on Therapy to Corticosteroids for Children and Adults with Asthma: a Systematic Review. Chest 2010; [Epub ahead of print].
36. Prussin C, Griffith D T, Boesel K M, Lin H, Foster B, Casale T B. Omalizumab treatment downregulates dendritic cell Fc epsilonRI expression. J Allergy Clin Immunol 2003; 112:1147-54.
37. Maurer D, Ebner C, Reininger B, Fiebiger E, Kraft D, Kinet J P, Stingl. G. The high affinity IgE receptor (Fc epsilon RI) mediates IgE-dependent allergen presentation. J Immunol. 1995 Jun. 15; 154(12):6285-90.
38. Maurer D, Fiebiger E, Reininger B, Ebner C, Petzelbauer P, Shi G P, Chapman H A, Stingl G. Fc epsilon receptor I on dendritic cells delivers IgE-bound multivalent antigens into a cathepsin S-dependent pathway of MHC class II presentation. J Immunol. 1998 Sep. 15; 161(6):2731-9.
39. Ostrowski M, Carmo N B, Krumeich S, Fanget I, Raposo G, Savina A, Moita C F, Schauer K, Hume A N, Freitas R P, Goud B, Benaroch P, Hacohen N, Fukuda M, Desnos C, Seabra M C, Darchen F, Amigorena S, Moita L F, Thery C. Rab27a and Rab27b control different steps of the exosome secretion pathway. Nat Cell Biol. 2010 January; 12(1):19-30; sup pp 1-13. Epub 2009 Dec. 6.
40. Maurer D, Fiebiger S, Ebner C, Reininger B, Fischer G F, Wichlas S, Jouvin M H, Schmitt-Egenolf M, Kraft D, Kinet J P, Stingl G. Peripheral blood dendritic cells express Fc epsilon RI as a complex composed of Fc epsilon RI alpha- and Fc epsilon RI gamma-chains and can use this receptor for IgE-mediated allergen presentation. J Immunol. 1996 Jul. 15; 157(2):607-16.

1. A method of diagnosing an IgE-mediated disorder in a subject, the method comprising:
(i) detecting or measuring a level of soluble FcεRI (sFcεRI) in a sample from a subject,
(ii) comparing the level of sFcεRI in the sample to a predetermined value, and,
(iii) if the level of sFcεRI in the sample is above the predetermined value, identifying the subject as having or being at risk of having an IgE-mediated disorder.

2. The method of claim 1, wherein the IgE-mediated disorder is selected from the group consisting of: esophagitis, gastroenteritis, hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis and necrotizing enterocolitis.

3. The method of claim 1, wherein the esophagitis is eosinophilic esophagitis (EoE).

4. The method of claim 1, wherein the gastroenteritis is eosinophilic gastroenteritis (EoG).

5. The method according to claim 1, wherein the subject is a human subject.

6. (canceled)

7. The method according to claim 1, wherein the sample is blood, serum, plasma, lymph, saliva or urine.

8. The method according to claim 1, wherein the subject has a normal level of serum IgE.

9-16. (canceled)

17. A method of evaluating the efficacy of a therapy for an IgE-mediated disorder in a subject, the method comprising:
(i) measuring a level of sFcεRI in a sample from a subject having or at risk of having an IgE-mediated disorder before a therapy for the disorder,
(ii) measuring a level of sFcεRI in a sample from a subject having or at risk of having an IgE-mediated disorder after the therapy for the disorder,
(iii) comparing the level of sFcεRI in the samples before and after the therapy, wherein a decrease in sFcεRI in the sample after the therapy relative to the sample before the therapy indicates that the subject is responsive to the therapy.

18. The method of claim 17, further comprising repeating steps (ii) and (iii) so as to monitor the efficacy of the therapy.

19. The method of claim 17, wherein the IgE-mediated disorder is selected from the group consisting of: esophagitis, gastroenteritis, hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis and necrotizing enterocolitis.

20. The method of claim 19, wherein the esophagitis is eosinophilic esophagitis (EoE).

21. The method of claim 19, wherein the gastroenteritis is eosinophilic gastroenteritis (EoG).

22. The method according to claim 17, wherein the subject is a human subject.

23. (canceled)

24. The method according to claim 17, wherein the sample is blood, serum, plasma, lymph, saliva or urine.

25. The method according to claim 17, wherein the subject has a normal level of serum IgE.

26. A method of evaluating responsiveness to an immunotherapy in a subject, the method comprising:
(i) measuring a level of sFcεRI in a sample from a subject in need of an immunotherapy collected before the immunotherapy,
(ii) measuring a level of sFcεRI in a biological sample collected from the subject after the immunotherapy,
(iii) comparing sFcεRI levels in the samples collected before and after the therapy,
wherein an increase in the level of sFcεRI in the sample collected after the immunotherapy relative to the sample collected before the immunotherapy indicates that the subject is responsive to the immunotherapy.

27. The method of claim 26, wherein the subject has a cancer.

28. The method of claim 26, wherein the immunotherapy is a cancer immunotherapy.

29. A method of treating an IgE-mediated disorder in a subject, the method comprising:
administering a composition comprising sFcεRI to a subject having or at risk of having an IgE-mediated disorder in an amount effective to treat the disorder.

30. The method of claim 29, wherein the composition further comprises a pharmaceutically acceptable carrier.

31. The method of claim 29, wherein the IgE-mediated disorder is selected from the group consisting of: esophagitis, gastroenteritis, hypersensitivity, eczema, urticaria, allergic bronchopulmonary aspergillosis, parasitic diseases, interstitial cystitis, hyper-IgE syndrome, ataxia-telangiectasia, Wiskott-Aldrich syndrome, thymic alymphoplasia, IgE myeloma, graft-versus-host reaction, allergic rhinitis, asthma, allergic asthma, atopic dermatitis, allergic gastroenteropathy, Churg-Strauss Syndrome, enteritis, gastroenteropathy, glioma, ovarian cancer, leukemia, inflammatory bowel disease, mucositis and necrotizing enterocolitis.

32. An assay for detecting sFcεRI in a sample, the assay comprising:
an agent that binds to sFcεRI, and
a solid substrate,
wherein the agent is immobilized on the solid substrate, and
wherein the sFcεRI is detected with a probe.

33. The assay of claim 32, wherein the agent is a recombinant IgE.

34-39. (canceled)
